(19) United States
(12) Patent Application Publication
(10) Pub. No.: US 2020/0155583 A1
(43) Pub. Date: May 21, 2020

(54) SOLID ORAL FORMULATIONS OF AMPHOTERICIN B

(71) Applicant: iCo Therapeutics Inc., Vancouver (CA)

(72) Inventors: Peter HNIK, Vancouver (CA); Roch THIBERT, Laval. Quebec (CA); Aimesther BETANCOURT, Laval, Quebec (CA)

(21) Appl. No.: 16/487,350

(22) PCT Filed: Feb. 21, 2018
(86) PCT No.: PCT/US18/18961

§ 371 Date: Aug. 20, 2019

Related U.S. Application Data
(60) Provisional application No. 62/461,427, filed on Feb. 21, 2017.

(51) Int. Cl. A61K 31/7048; A61K 9/16

(52) U.S. Cl. CPC A61K 31/7048; A61K 9/1676

(57) ABSTRACT

The present disclosure describes solid dosage forms comprising amphotericin B. Also described herein are methods of treating fungal infections and Lesishmania infections.

### CROSS-REFERENCE TO RELATED APPLICATIONS

This application claims priority to U.S. Provisional Application No. 62/461,427, filed on Feb. 21, 2017, which is incorporated by reference herein in its entirety.

### BACKGROUND

Amphotericin B is an effective antifungal agent and is the drug of choice for treating serious systemic fungal infections and Lesishmania infections. However, amphotericin B has several unfavorable properties which severely impede its use as a therapeutic agent. First, amphotericin B is insoluble in water. Second, amphotericin B cannot be absorbed in the gastrointestinal tract (GIT). Third, amphotericin B is not stable in the acid environment of the stomach. Each of these properties limits the bioavailability of amphotericin B.

To overcome the above problems which result in limited bioavailability, amphotericin B was administered in a liposomal composition (Ampbisome®) or as colloidal dispersion (Fungizone®, Abelcet®). However, intravenous injection and infusion of amphotericin B have significant disadvantages. First, the intravenous injection and infusion of amphotericin B have been associated with considerable side effects such as fever, chills, bone pain, nephrotoxicity, and thrombophlebitis. Second, intravenous amphotericin B must be administered over 30-40 days, and thus this dosing regimen is expensive and suffers from low patient compliance. These drawbacks are particularly issues in developing countries where Lesishmania infections occur.

U.S. Pat. Nos. 8,592,382 and 8,673,866 describe orally administered liquid formulations comprising amphotericin B and a mixture of fatty acid glycerol esters and polyethylene oxide-containing fatty acid esters. The fatty acid glycerol esters and polyethylene oxide-containing fatty acid esters are present in substantial excess (greater than 180:1) relative to amphotericin B, which was described as critical to achieving bioavailability of amphotericin B in an oral dosage form. However, the large amount of oily components in these formulations may cause gastric upset, such as nausea and diarrhea which limits patient compliance, particularly since an extended dosing regime is required. In addition, dosing such liquid suspensions is messy and can result in under or overdosing due to dispensing errors, spillage, and/or losses of residual formulation remaining in the dispensing device. There is thus a need to provide stable bioavailable dosage forms of amphotericin B, ideally solid dosage forms, which do not exhibit the limitations of known amphotericin B formulations.

The present disclosure provides a solid dosage form which overcomes the limitations of the conventional amphotericin B compositions.

### SUMMARY

The disclosure, in various embodiments, is directed to solid dosage forms (e.g., solid or semi-solid dosage forms) comprising lipophilic drugs, for example amphotericin B. In embodiments, the solid dosage forms disclosed herein achieve bioavailability equivalent to liquid formulations commonly used to administer amphotericin B.

In some embodiments, the solid dosage form comprises amphotericin B and at least one lipophilic component which are coated on a solid carrier. In other embodiments, the % w/w of amphotericin B in the solid dosage form is greater than a % w/w of the at least one lipophilic component. In further embodiments, the % w/w of amphotericin B is in the range of about 20% to about 30% of the total weight of the solid dosage form.

In some embodiments, amphotericin B is present in the solid dosage form in an amount in the range of from about 50 mg to about 200 mg. In other embodiments, amphotericin B is present in amount of about 100 mg. In still other embodiments, wherein the amphotericin B is present in amount of about 150 mg.

In some embodiments, the at least one lipophilic component is selected from the group consisting of a polyethylene oxide-containing fatty acid ester, fatty acid glycerol ester, and a combination thereof.

In some embodiments, the solid carrier is a bead or a saccharide. In other embodiments, the disclosure provides for a capsule comprising a solid dosage form described herein.

In some embodiments, the present disclosure provides for a method of treating leishmaniasis comprising administering an effective amount of a solid dosage form described herein.

### DESCRIPTION OF THE DRAWINGS

FIG. 1 illustrates the preparation of Amphotericin B/Gelucire/Peceol/TPGS/Powdered excipients formulations.

FIG. 2 shows thermogravimetric analysis (TGA) curves of Amphotericin B (23%) for Formulations 1-3.

FIG. 3 shows the dissolution profiles of Amphotericin B for Formulations 1-3.

FIG. 4 shows the dissolution profiles of Amphotericin B in scale-up Formulation 1A and Formulation 1B at T=0/Initial compared to Formulation 1, Formulation 2.

FIG. 5 shows the dissolution profile in 0.5% SDS in water of solid and semi-solid Amphotericin B formulations in capsules.

FIG. 6 shows the dissolution profile in FeSSIF pH 5.8 of solid and semi-solid Amphotericin B formulations in capsules.

FIG. 7 shows the dissolution profiles of 100 mg capsules comprising lipid based formulations.

FIG. 8 shows the dissolution profile of Amphotericin B granular formulations in capsules at T=0 and under stability storage conditions.

FIG. 9 shows the dissolution profile of Amphotericin B lipid based capsules of Formula 5A at T=0 and under stability storage conditions.

FIG. 10 shows the tissue concentrations of Amphotericin B measured in a dog model.

FIG. 11 shows the blood plasma concentrations of Amphotericin B measured for Formulation 1A (A), Formulation B (B), and the conventional lipid formulation (C).

FIG. 12 shows individual plasma levels of amphotericin B following oral dosing of dogs with 500 mg of Amphotericin B in Formulation 1A.

FIG. 13 shows mean plasma levels of amphotericin B following oral dosing of dogs with 500 mg of amphotericin B in Formulation 1A.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

The term “pharmaceutically acceptable” means biologically or pharmacologically compatible for in-vivo use in animals or humans, and can mean approved by a regulatory agency of the Federal or a state government or listed in the U.S. Pharmacopeia or other generally recognized pharmacopeia for use in animals, and more particularly in humans.

The term “subject,” as used herein, comprises any and all organisms and includes the term “patient.” “Subject” may refer to a human or any other animal.

The term “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

As used herein, the term “about,” when located before a dosage amount or dosage range of a specific ingredient, refers to an amount or range closely above and/or closely below the stated amount or range that does not manifestly alter the therapeutic effect of the specific ingredient from the stated amount or range.

Any lipophilic therapeutic agent may be formulated using the solid dosage form disclosed herein. For example, specific therapeutic agents that can be administered using the formulation and methods disclosed herein include tetracycline, doxycycline, oxytetracycline, chloramphenicol, erythromycin, acyclovir, idoxuridine, tromantadine, miconazole, ketoconazole, fluconazole, itraconazole, econazole, griseofulvin, amphotericin B, nystatine, metronidazole, metronidazole benzoate, tinidazole, indomethacin, ibuprofen, piroxicam, diclofenac, disodium cromoglycate, nitroglycerin, isosorbide dinitrate, verapamil, nifedipine, diltiazem, digoxine, morphine, cyclosporins, buprenorphine, lidocaine, diazepam, nitrazepam, flurazepam, estazolam, flunitrazepam, triazolam, alprazolam, midazolam, temazepam lormetazepam, brotizolam, clobazam, clonazepam, lorazepam, oxazepam, busiprone, sumatriptan, ergotamine derivatives, cinnarizine, anti-histamines, ondansetron, tropisetron, granisetrone, metoclopramide, disulfuram, vitamin K, paclitaxel, docetaxel, camptothecin, SN38, cisplatin, carboplatin, efavirenz, saquinavir, ritonavir, and clofazamine.

In particular embodiments, the solid dosage form comprises amphotericin B. In further embodiments, the amphotericin B solid dosage forms of the present disclosure can further include a second therapeutic agent, for example any of those disclosed herein.

In embodiments, the bioavailability of amphotericin B in the solid dosage forms described herein is at least equivalent to conventional liquid formulations, such as those disclosed in U.S. Pat. Nos. 8,592,382 and 8,673,866, each of which are herein incorporated by reference in its entirety for all purposes. For example, the amphotericin B formulation disclosed in U.S. Pat. No. 8,673,866 utilizes an isotropic mixture of lipophilic components (oils, surfactants, solvents, and co-solvents/surfactants) at a weight ratio relative to amphotericin B exceeding about 189 to 1 to achieve suitable levels of bioavailability, which resulted in an oily formulation that causes gastric upset. The present inventors surprisingly and unexpectedly discovered that equivalent levels of bioavailability can be achieved with solid dosage forms comprising a significantly reduced amount of the lipophilic components, without causing gastric upset.

In some embodiments, the solid dosage forms of the present disclosure provide equivalent bioavailability to the above-referenced conventional liquid formulations, with a large ratio of amphotericin B relative to one or more lipophilic components of the formulation, whereas conventional liquid amphotericin formulations employ a large ratio of lipophilic components to amphotericin B in order to provide sufficient bioavailability. In embodiments, the solid compositions of the present disclosure have a weight ratio of amphotericin B to the lipophilic components in the range of about 100:1 to about 1:1, for example about 100:1, about 95:1, about 90:1, about 85:1, about 80:1, about 75:1, about 70:1, about 65:1, about 60:1, about 55:about 50:1, about 45:1 about 40:1, about 35:1, about 30:1, about 25:1, about 20:1, about 15:1, about 10:1, about 9.5:1 about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2.5:1, about 2:1, about 1.5:1, or about 1:1, inclusive of all ranges and subranges therebetween.

In other embodiments, the solid dosage forms of the present disclosure provide equivalent bioavailability to the above-referenced liquid formulations and have a smaller excess of the lipophilic components relative to amphotericin B compared to the conventional liquid formulations. In embodiments, the weight ratio of the one or more lipophilic components (e.g., one, two, three, etc., lipophilic components) in the solid dosage forms of the present disclosure to the amphotericin B is in the range of about 100:1 to about 1:1, for example about 100:1, about 95:1, about 90:1, about 85:1, about 80:1, about 75:1, about 70:1, about 65:1, about 60:1, about 55:1 about 50:1, about 45:1 about 40:1, about 35:1, about 30:1, about 25:1, about 20:1, about 15:1, about 10:1, about 9.5:1 about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2.5:1, about 2:1, about 1.5:1, or about 1:1, inclusive of all ranges and subranges therebetween.

In alternative embodiments, the solid dosage forms of the present disclosure comprise about 10-30 weight % amphotericin B and about 1-10 weight % (total) of the one or more lipophilic components. For example, the weight % amphotericin B is about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%, inclusive of all ranges and subranges therebetween; and the total weight % lipophilic components is about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10%, inclusive of all ranges and subranges therebetween.

In some embodiments, at least one lipophilic component is used in combination with the therapeutic agent (e.g. amphotericin B). In other embodiments, at least one lipophilic component is used to facilitate coating the therapeutic agent onto a solid carrier. The lipophilic component may include any hydrophobic material in which the therapeutic agent (e.g. amphotericin B) can be dissolved or suspended, and which is pharmaceutically acceptable. Lipophilic components used to solubilize the therapeutic agent may be selected based on the hydrophilic-lipophilic balance (HLB) of the therapeutic agent and the lipophilic component, or of the lipid and an optional organic solvent to facilitate solubilization of the amphotericin B in the lipophilic component. Suitable lipid materials for solubilizing the therapeutic agent (e.g. amphotericin B) may have an HLB value which is equal to that of the therapeutic agent or otherwise sufficient to solubilize the therapeutic agent in an appropriate solvent. For example, lipophilic components suitable to solubilize amphotericin B in ethanol may have an HLB of 14 or less (e.g., 13, 12, 11, or 10).

Each lipophilic component in the compositions of the present disclosure can be selected from natural (human-, animal-, or plant-derived) or synthetic sources. The lipophilic component can be a liquid or a solid at room temperature, provided that the solid can be melted upon heating and the melted lipophilic component does not degrade or denature the therapeutic agent (e.g. amphotericin B). In some embodiments, at least one lipophilic component may be used to solubilize the therapeutic agent (e.g. a lipophilic drug, e.g. amphotericin B). In other embodiments, the lipophilic component may be selected to improve the oral absorption of the therapeutic agent (amphotericin B). In further embodiments, the lipophilic component may be selected to improve the bioavailability of the therapeutic agent (e.g. amphotericin B). In still other embodiments, the lipophilic component may include a surfactant. In some such embodiments, the lipophilic component may be a non-ionic surfactant. In even further embodiments, the lipophilic component is a lipophilic binder material which promotes coating or adhesion of the therapeutic agent to a solid carrier.

In embodiments, the dosage forms disclosed herein may include one lipophilic component or a mixture of two or more lipophilic components (e.g., a mixture of 3 lipophilic components, 4 lipophilic components, 5 lipophilic components, etc.). In embodiments which entail two lipophilic components, the weight ratio of the first lipophilic component to the second lipophilic component is in the range of about 99:1 to about 1:99, for example about 99:1, about 95:5, about 90:10, about 85:15, about 80:20, about 75:25, about 70:30, about 65:35, about 60:40, about 55:45 about 50:50, about 45:55 about 40:60, about 35:65, about 30:70, about 25:75, about 20:80, about 15:85, about 10:90, about 5:95 and about 1:99, inclusive of all ranges and subranges therebetween.

Non-limiting examples of lipophilic components which are useful in the solid dosage forms disclosed herein include pharmaceutically acceptable fats, fatty substances, oils, phospholipids, sterols, and waxes. Fats generally refer to esters of glycerol (e.g., mono-, di- or triesters of glycerol and fatty acids). Suitable fats and fatty substances include but not limited to fatty alcohols (such as lauryl, myristyl, stearyl, cetyl or cetostearyl alcohol, etc.), fatty acids and derivatives, including but not limited to fatty acid esters, fatty acid glycerides (mono-, di- and triglycerides), and hydrogenated fats. Fats may be either solid or liquid at normal room temperature, depending on their structure and composition.

Suitable oils include pharmaceutically acceptable animal (e.g., fatty acid esters), mineral (e.g., paraffin oils), vegetable (e.g., vegetable oils), or synthetic hydrocarbons that are liquid at room temperature. Examples of pharmaceutically acceptable oils include but are not limited to: mineral oils such as paraffin oils; vegetable oils such as castor oils, hydrogenated vegetable oil, sesame oils, and peanut oils; and animal oils and fats such as triglycerides and butters. Partially hydrogenated vegetable oils are derived from natural products and generally comprise a mixture of glycerides of C₁₄₋₂₀ fatty acids, in particular palmitic and stearic acids. Suitable examples of partially hydrogenated vegetable oils include partially hydrogenated cottonseed oil, soybean oil, corn oil, peanut oil, palm oil, sunflower seed oil or mixtures thereof. Chemical equivalents of partially hydrogenated vegetable oils include synthetically produced glycerides of C₁₄₋₂₀ fatty acids having the same properties as the naturally derived products as hereinbefore described.

Suitable phospholipids include pharmaceutically acceptable plant, animal, and synthetic phospholipids. Examples of pharmaceutically acceptable phospholipids include cholines phosphatidylethanolamine, and phosphatidylglycerols, such as, but not limited to, phosphatidylcholine, 1,2-dierucoylphosphatidylcholine, 1,2-dimyristoylphosphatidylcholine, 1,2-dioleoylphosphatidylcholine, 1,2-dioleoylphosphatidylserine, 1,2-di stearoylphosphatidylglycerol, 1,2-dipalmitoylphosphatidylcholine, 1,2-di stearoylphosphatidylcholine, 1,2-di stearoylphosphatidylglycerol, egg phosphatidylcholine, egg phosphatidylglycerol, soy phosphatidylcholine, glycerophosphocholine, hydrogenated soybean phosphatidylcholine, lysophosphatidylcholine, lysophosphatidylethanolamine, N-(carbonyl-methoxypolyethylene glycol 2000)-1,2-distearoylphosphatidylethanolamine sodium salt, muramyltripeptide-phosphatidylethanolamine, 1-palmitoyl-2-linoleoylphosphatidylcholine, 1-palmitoyl-2-linoleoylphosphatidylglycerol, 1-palmitoyl-2-oleoylphosphatidylcholine, 1-palmitoyl-2-oleoylphosphatidylglycerol, polyenylphosphatidylcholine, 1-palmitoyl-2-stearoylphosphatidylcholine, 1-palmitoyl-2-stearoylphosphatidylglycerol, 1-stearoyl-2-linoleoylphosphatidylcholine, 1-stearoyl-2-linoleoylphosphatidylglycerol, sphingomyelin, 1-stearoyl-2-oleoyl phosphatidylcholine, 1-stearoyl-2-oleoyl phosphatidylglycerol, and the like.

Suitable waxes include animal waxes, plant waxes, mineral waxes, and petroleum waxes. Examples of waxes include, but are not limited to, glyceryl behenate, glyceryl monostearate, stearic acid, palmitic acid, lauric acid, carnauba wax, cetyl alcohol, glyceryl stearate beeswax, paraffin wax, ozokerite, candelilla wax, cetyl alcohol, stearyl alcohol, spermaceti, carnauba wax, bayberry wax, montan, ceresin, and microcrystalline waxes.

In particular embodiments, lipophilic components suitable for use in the solid dosage forms disclosed herein include fatty acid glycerol esters, polyethylene oxide-containing fatty acid esters, and combinations thereof.

In specific embodiments, the amphotericin B formulations of the present disclosure include one or more fatty acid glycerol esters. As used herein the term “fatty acid glycerol esters” refers to esters formed between glycerol and one or more fatty acids including mono-, di-, and tri-esters (i.e., glycerides). Suitable fatty acids include saturated and unsaturated fatty acids having from eight (8) to twenty-two (22) carbons atoms (i.e., C8-C22 fatty acids). In certain embodiments, suitable fatty acids include C12-C18 fatty acids. The fatty acid glycerol esters useful in the formulations can be provided by commercially available sources. A representative source for the fatty acid glycerol esters is a mixture of mono-, di-, and triesters commercially available as PECEOL® (Gattefosse, Saint Priest Cedex, France), commonly referred to as “glyceryl oleate” or “glyceryl monooleate.” In some embodiments, when PECEOL® is used as the source of fatty acid glycerol esters in the formulations, the fatty acid glycerol esters comprise from about 32 to about 52% by weight fatty acid monoglycerides, from about 30 to about 50% by weight fatty acid diglycerides, and from about 5 to about 20% by weight fatty acid triglycerides. The fatty acid glycerol esters comprise greater than about 60% by weight oleic acid (C18:1) mono-, di-, and triglycerides. Other fatty acid glycerol esters include esters of palmitic acid (C16) (less than about 12%), stearic acid (C18) (less than about 6%), linoleic acid (C18:2) (less than about 35%), linolenic acid (C18:3) (less than about 2%), arachidic acid (C20) (less than about 2%), and eicosanoic acid (C20:1) (less than about 2%). PECEOL® can also include free glycerol (typically about 1%). In one embodiment, the fatty acid glycerol esters comprise about 44% by weight fatty acid monoglycerides, about 45% by weight fatty acid diglycerides, and about 9% by weight fatty acid triglycerides, and the fatty acid glycerol esters comprise about 75% by weight oleic acid (C18:1) mono-, di-, and triglycerides. Other fatty acid glycerol esters include esters of palmitic acid (C16) (about 4%), stearic acid (CI5) (about 2%), linoleic acid (CIS:2) (about 12%), linolenic acid (C18:3) (less than 1%), arachidic acid (C20) (less than 1%), and eicosanoic acid (C20:1) (less than 1%).

In embodiments, a fatty acid glycerol ester may be the sole lipid in the amphotericin B formulation. In other embodiments, the formulation may include a mixture fatty acid glycerol ester, for example any of those disclosed herein. In still other embodiments, one or more fatty acid glycerol ester may be used in combination with other lipophilic components as described herein, such one or more polyethylene oxide-containing fatty acid esters as described herein.

In some embodiments, the amphotericin B formulations described herein comprise at least one polyethylene oxide-containing lipophilic components, such as fatty acid esters. As used herein, the term “polyethylene oxide-containing fatty acid ester” refers to a fatty acid ester that includes a polyethylene oxide group (i.e., polyethylene glycol group) covalently coupled to the fatty acid through an ester bond. Polyethylene oxide-containing fatty acid esters include mono- and di-fatty acid esters of polyethylene glycol. Suitable polyethylene oxide-containing fatty acid esters are derived from fatty acids including saturated and unsaturated fatty acids having from eight (8) to twenty-two (22) carbons atoms (i.e., a polyethylene oxide ester of a C8-C22 fatty acid). In certain embodiments, suitable polyethylene oxide-containing fatty acid esters are derived from fatty acids including saturated and unsaturated fatty acids having from twelve (12) to eighteen (18) carbons atoms (i.e., a polyethylene oxide ester of a C12-C18 fatty acid). Representative polyethylene oxide-containing fatty acid esters include saturated C8-C22 fatty acid esters. In certain embodiments, suitable polyethylene oxide-containing fatty acid esters include saturated C12-C18 fatty acids.

The molecular weight of the polyethylene oxide group of the polyethylene oxide-containing fatty acid ester can be varied to optimize the solubility of the therapeutic agent (e.g., amphotericin B) in the formulation. Representative average molecular weights for the polyethylene oxide groups can be from about 350 to about 2000. In one embodiment, the average molecular weight for the polyethylene oxide group is about 1500.

In some embodiments, when the amphotericin B formulation includes a polyethylene oxide-containing fatty acid in the lipophilic component, the lipophilic component may include only one type of polyethylene oxide-containing fatty acid. In other embodiments, the polyethylene oxide-containing fatty acid in the lipophilic component may include a mixture of polyethylene oxide-containing fatty acid esters (mono- and di-fatty acid esters of polyethylene glycol).

The polyethylene oxide-containing fatty acid esters useful in the formulations of the present disclosure can be provided by commercially available sources. Representative polyethylene oxide-containing fatty acid esters (mixtures of mono- and diesters) are commercially available under the designation GELUCIRE® (Gattefosse, Saint Priest Cedex, France). Suitable polyethylene oxide-containing fatty acid esters include GELUCIRE® 44/14, GELUCIRE® 50/13, GELUCIRE® 53/10, and GELUCIRE® 48/16. The numerals in these designations refer to the melting point and hydrophilic/lipophilic balance (HLB) of these materials, respectively. GELUCIRE® 44/14, GELUCIRE 50/13, GELUCIRE® 53/10, and GELUCIRE® 48/16 are mixtures of (a) mono-, di-, and triesters of glycerol (glycerides) and (b) mono- and diesters of polyethylene glycol (macrogols). The GELUCIRES can also include free polyethylene glycol (e.g., PEG 1500).

Lauric acid (C12) is the predominant fatty acid component of the glycerides and polyethylene glycol esters in GELUCIRE® 44/14. GELUCIRE® 44/14 is referred to as a mixture of glyceryl dilaurate (lauric acid diester with glycerol) and PEG dilaurate (lauric acid diester with polyethylene glycol), and is commonly known as PEG-32 glyceryllaurate (Gattefosse) lauroyl macrogol-32 glycerides EP, or lauroyl polyoxylglycerides USP/NF. GELUCIRE® 44/14 is produced by the reaction of hydrogenated palm kernel oil with polyethylene glycol (average molecular weight 1500). GELUCIRE® 44/14 includes about 20% mono-, di- and, triglycerides, about 72% mono- and di-fatty acid esters of polyethylene glycol 1500, and about 8% polyethylene glycol 1500.

GELUCIRE® 44/14 includes lauric acid (C12) esters (30 to 50%), myristic acid (C14) esters (5 to 25%), palmitic acid (C16) esters (4 to 25%), stearic acid (C18) esters (5 to 35%), caprylic acid (C8) esters (less than 15%), and capric acid (C10) esters (less than 12%). GELUCIRE® 44/14 may also include free glycerol (typically less than about I %). In a representative formulation, GELUCIRE® 44/14 includes lauric acid (C12) esters (about 47%), myristic acid (C14) esters (about 18%), palmitic acid (C16) esters (about 10%), stearic acid (C18) esters (about 11%), caprylic acid (C8) esters (about 8%), and capric acid (C10) esters (about 12%).

Palmitic acid (C16) (40-50%) and stearic acid (C18) (48-58%) are the predominant fatty acid components of the glycerides and polyethylene glycol esters in GELUCIRE® 50/13. GELUCIRE® 50/13 is known as PEG-32 glyceryl palmitostearate (Gattefosse), stearoyl macrogolglycerides EP, or stearoyl polyoxylglycerides USP/NF). GELUCIRE® 50/13 includes palmitic acid (C16) esters (40 to 50%), stearic acid (C18) esters (48 to 58%) (stearic and palmitic acid esters greater than about 90%), lauric acid (C12) esters (less than 5%), myristic acid (C14) esters (less than 5%), caprylic acid (C8) esters (less than 3%), and capric acid (C10) esters (less than 3%). GELUCIRE® 50/13 may also include free glycerol (typically less than about 1%). In a representative formulation, GELUCIRE® 50/13 includes palmitic acid (C16) esters (about 43%), stearic acid (CIS) esters (about 54%) (stearic and palmitic acid esters about 97%), lauric acid (C12) esters (less than 1%), myristic acid (C14) esters (about 1%), caprylic acid (C8) esters (less than 1%), and capric acid (C10) esters (less than 1%) Stearic acid (C18) is the predominant fatty acid component of the glycerides and polyethylene glycol esters in GELUCIRE® 53/10. GELUCIRE® 53/10 is known as PEG-32 glyceryl stearate (Gattefosse).

In one embodiment, the polyethylene oxide-containing fatty acid ester is a lauric acid ester, a palmitic acid ester, or a stearic acid ester (i.e., mono- and di-lauric acid esters of polyethylene glycol, mono- and di-palmitic acid esters of polyethylene glycol, mono- and di-stearic acid esters of polyethylene glycol). Mixtures of these esters can also be used.

In some embodiments, the solid dosage form comprises at least one fatty acid glycerol ester and at least one polyethylene oxide-containing fatty acid ester. In such embodiments, the ratio of the at least one fatty acid glycerol ester to the at least one polyethylene oxide-containing fatty acid ester is in the range of from about 90:10 to about 10:90, including about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, or about 10:90, inclusive of all ranges and subranges therebetween. In further embodiments, the solid dosage form comprises PECEOL® and GELUCIRE® 44/14 (as described herein). In embodiments, the ratio of PECEOL® and GELUCIRE® 44/14 is in the range of from about 90:10 to about 10:90, including about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, or about 10:90, inclusive of all ranges and subranges therebetween.

The amphotericin B formulations disclosed herein optionally include a stabilizer. In some embodiments, the stabilizer is a thermal stabilizer, for example tocopherol polyethylene glycol succinate (e.g., TPGS or vitamin E TPGS). In some embodiments, the stabilizer is an antioxidant, such as butylated hydroxyanisole (BHA) or butylated hydroxytoluene (BHT). Such thermal stabilizers and/or antioxidants enhance the thermal stability of the formulation, which in turn, can increase the formulation's shelf-life, which is particularly important in tropical regions of the world where prolonged exposure to high temperatures are common and refrigerated medicinal storage is difficult.

Structurally, tocopherol polyethylene glycol succinates have a polyethylene glycol (PEG) covalently coupled to tocopherol (e.g., a-tocopherol or vitamin E) through a succinate linker. Because PEG is a polymer, a variety of polymer molecular weights can be used to prepare the TPGS. In one embodiment, the TPGS is tocopherol polyethylene glycol succinate 1000, in which the average molecular weight of the PEG is 1000. One suitable tocopherol polyethylene glycol succinate is vitamin E TPGS commercially available from Eastman.

In some embodiments, the solid dosage forms of the present disclosure comprise a dosage of amphotericin B in the range of from about 1 mg to about 500 mg, including about 1 mg, about 5 mg, about 10 mg, 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 g, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg, inclusive of all ranges and subranges therebetween.

In some embodiments, the % w/w of amphotericin B in the solid dosage form is at least about 1%, or at least about 5%, or about at least about 10%, or at least about 15%, or about 20%, or at least about 25%, or at least about 30%, or at least about 35%, or at least about 40%, or at least about 45%, or at least about 50%, or at least about 55%, or at least about 60%, or at least about 65%, or at least about 70%. In some embodiments, the % w/w of amphotericin B in the solid dosage form is in the range of about 1% to about 70%, or about 5% to about 70%, or about 5% to about 60%, or about 5% to about 50%, or about 5% to about 40%, or about 10% to about 40%, or about 15% to about 40%, or about 20% to about 40%, or about 20% to about 40%, or about 20% to about 35%, or about 20% to about 30%.

The solid dosage forms of the present disclosure can be prepared by any suitable method, including granulation of the therapeutic agent (e.g. amphotericin B) with excipients (e.g. fillers, glidants, lubricants, etc. known in the art and described herein), extrusion of the therapeutic agent with excipients, direct compression of the therapeutic agent with excipients to form tablets, etc.

In particular embodiments, the solid dosage forms the present disclosure can be prepared by coating the active agent, e.g. amphotericin B on a solid carrier. The solid carrier can be any material upon which a drug-containing composition can be coated and which is suitable for human consumption. Any conventional coating process can be used. For example, the therapeutic agent, e.g. amphotericin B can be dissolved or suspended in a suitable solvent (e.g., ethanol), together with an optional binder, or alternatively one or more of the lipophilic components described herein, and deposited on the solid carrier by methods known in the art, e.g. fluidized bed coating or pan coating methods. The solvent can be removed e.g. by drying, or in situ during the coating process (e.g., during fluidized bed coating), and/or in a subsequent drying step.

In some embodiments, the solid carrier may be an inert bead or an inert particle. In other embodiments, the solid carrier a non-pareil seed, an acidic buffer crystal, an alkaline buffer crystal, or an encapsulated buffer crystal.

In some embodiments, the solid carrier may be a sugar sphere, cellulose sphere, lactose sphere, lactose-microcrystalline (MCC) sphere, mannitol-MCC sphere, or silicon dioxide sphere.

In other embodiments, the solid carrier may be a saccharide, a sugar alcohol, or combinations thereof. Suitable saccharides include lactose, sucrose, maltose, and combinations thereof. Suitable sugar alcohols include mannitol, sorbitol, xylitol, maltitol, arabitol, ribitol, dulcitol, iditol, isomalt, lactitol, erythritol and combinations thereof.

In embodiments, the solid carrier may be formed by combining any of the above with a filler. Examples of suitable fillers which may be used to form a solid carrier include lactose, microcrystalline cellulose, silicified microcrystalline cellulose, mannitol-microcrystalline cellulose and silicon dioxide.

In other embodiments, the dosage form disclosed herein does not include a solid carrier.

In embodiments, the solid dosage forms disclosed herein can include one or more pharmaceutically acceptable excipients. Pharmaceutically acceptable excipients include fillers, diluents, glidants, disintegrants, binders and lubricants. Other pharmaceutically acceptable excipients include acidifying agents, alkalizing agents, preservatives, antioxidants, buffering agents, chelating agents, coloring agents, complexing agents, emulsifying and/or solubilizing agents, flavors, perfumes, humectants, sweetening agents and wetting agents.

Examples of suitable fillers and/or binders include lactose (e.g. spray-dried lactose, a-lactose, P-lactose, Tabletose®, various grades of Pharmatose®, Microtose® or FastFlo®), microcrystalline cellulose (various grades of Avicel®, Ceolus®, Elcema®, Vivacel®, Ming Tai® or Solka-Floc®), hydroxypropylcellulose, L-hydroxypropylcellulose (low substituted), low molecular weight hydroxypropyl methylcellulose (HPMC) (e.g. Methocel E, F and K from Dow Chemical, Metolose SH from Shin-Etsu, Ltd), hydroxyethylcellulose, sodium carboxymethylcellulose, carboxymethylhydroxyethylcellulose and other cellulose derivatives, sucrose, agarose, sorbitol, mannitol, xylitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, polyvinylpyrrolidone, and polyethylene glycol.

Examples of pharmaceutically acceptable diluents include calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose and sugar.

Pharmaceutically acceptable disintegrants include alginic acid, carboxymethylcellulose calcium, carboxymethylcellulose sodium (e.g., AC-DI-SOL® and Primellose®), colloidal silicon dioxide, croscarmellose sodium, crospovidone (e.g., Kollidon® and Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), potato starch, and starch.

Examples of pharmaceutically acceptable glidants include colloidal silicon dioxide, magnesium trisilicate, powdered cellulose, starch, talc, and tribasic calcium phosphate.

Pharmaceutically acceptable lubricants include stearic acid, magnesium stearate, calcium stearate or other metallic stearates (e.g., zinc stearate), glyceryl monostearate, glyceryl palmitostearate, waxes and glycerides, hydrogenated castor oil, hydrogenated vegetable oil, light mineral oil, polyethylene glycol, glyceryl behenate, colloidal silica, hydrogenated vegetable oils, corn starch, sodium lauryl sulfate, sodium stearyl fumarate, polyethylene glycols, alkyl sulfates, sodium benzoate, talc, and sodium acetate.

Flavoring agents and flavor enhancers make the dosage form more palatable to the patient. Common flavoring agents and flavor enhancers for pharmaceutical products that may be included in the composition and/or combination of the present invention include maltol, vanillin, ethyl vanillin, menthol, citric acid, fumaric acid, ethyl maltol, and tartaric acid.

Solid compositions may also be dyed using any pharmaceutically acceptable colorant to improve their appearance and/or facilitate patient identification of the product and unit dosage level.

The compositions disclosed herein can be formulated as a solid dosage form. Suitable solid dosage forms include tablets and capsules, such as a gelatin capsule or suitable synthetic capsules known in the art, such as HPMC (hydroxypropyl methylcellulose) capsules.

In embodiments, the solid dosage form described herein may be made by:
(a) dissolving at least one lipid and the therapeutic agent in a solvent, thereby forming a liquid mixture comprising the therapeutic agent;
(b) coating the mixture comprising a therapeutic agent on a solid carrier; and
(c) removing the solvent;

thereby forming drug coated-particles.

Any solvent in which the lipophilic component and the therapeutic agent can be dissolved can be used to make the solid dosage forms described herein. Examples of suitable solvents include lipophilic solvents, such as lipophilic organic solvents. Non-limiting examples of solvents include alcohols (e.g., ethanol, propanol, isopropanol, and the like), ketones (e.g., acetone and the like), dimethyl sulfoxide, dichloromethane, and the like.

The drug-coated particles can be milled as needed and passed through one or more mesh screens to produce granules having a desired size range. In various embodiments, the drug-coated particles may have an average particle size ranging from 10-2000 μm, e.g., 100-1000 μm, or 500-1000 μm.

In embodiments, the drug-coated particles can be filled into a capsule or compressed, optionally in combination with various excipients as described herein into a tablet.

In other embodiments, the therapeutic agent, e.g. amphotericin B, and an appropriate amount of a melt of room temperature solid lipophilic components (as described herein) can be mixed together (for example using methods, but not compositions disclosed in U.S. Pat. Nos. 8,592,382 and 8,673,866), optionally with a suitable amount of a solvent such as ethanol, until a homogeneous mixture or solution is formed. The resulting mixture or solution is then allowed to cool to thereby form a semi-solid composition. The semi-solid composition can then filled into a gelatin capsule to thereby provide a solid-dosage form.

In embodiments, the amphotericin B dosage forms disclosed herein are bioequivalent to conventional liquid formulations. That is, the solid dosage forms have an average maximum blood plasma concentration (Cₘₐₓ), an average AUC, and an average Tmax which is within the about 80% to about 125% of each of the average Cₘₐₓ, average AUC, and average Tₘₐₓ of conventional liquid compositions when administered to a human or animal, such as a rat model or beagle dog model. Cₘₐₓ, AUC, and Tₘₐₓ, as used herein, refer to the averages of such vales measured for a population of subjects.

Conventional liquid dosage forms provide a Cₘₐₓ of 71±10 ng/mL of amphotericin B when orally administered to a male Sprague Dawley rat at dosage of 4.5 mg/kg, or a Cₘₐₓ of 96±15 ng/mL of amphotericin B when orally administered to a male Sprague Dawley rat at dosage of 10 mg/kg.

In some embodiments, the solid dosage forms described herein provide a Cₘₐₓ within the range of about 80% to about 125% of 61 ng/mL to 81 ng/mL of amphotericin B (i.e., 71±10 ng/mL) when orally administered to a male Sprague Dawley rat at dosage of 4.5 mg/kg, e.g. about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 105 ng/mL, inclusive of all values and subranges therebetween.

In other embodiments, the solid dosage forms described herein provide a Cₘₐₓ within the range of about 80% to about 125% of 81 ng/mL to 111 ng/mL of amphotericin B (i.e., 96±15 ng/mL) when orally administered to a male Sprague Dawley rat at dosage of 10 mg/kg, e.g., about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 105 ng/mL, about 110 ng/mL, about 115 ng/mL, about 120 ng/mL, about 125 ng/mL, about 130 ng/mL, about 135 ng/mL, about 140 ng/mL, about 145 ng/mL, inclusive of all values and subranges therebetween.

Conventional liquid dosage forms provide an AUC₍₀₋₂₄₎ of 991±170 h·ng/mL of amphotericin B when orally administered to a male Sprague Dawley rat at dosage of 4.5 mg/kg, or a AUC₍₀₋₂₄₎ of 1534±229 h·ng/mL of amphotericin B when orally administered to a male Sprague Dawley rat at dosage of 10 mg/kg.

In some embodiments, the solid dosage forms described herein provide an AUC₍₀₋₂₄₎ of within the range of about 80% to about 125% of about 821 h·ng/mL to about 1161 h·ng/mL of amphotericin B (i.e., 991±170 h·ng/mL) when orally administered to a male Sprague Dawley rat at dosage of 4.5 mg/kg, e.g., about 600 h·ng/mL, about 650 h·ng/mL, about 700 h·ng/mL, about 750 h·ng/mL, about 800 h·ng/mL, about 850 h·ng/mL, about 900 h·ng/mL, about 950 h·ng/mL, about 1000 h·ng/mL, about 1050 h·ng/mL, about 1100 h·ng/mL, about 1150 h·ng/mL, about 1200 h·ng/mL, about 1250 h·ng/mL, about 1300 h·ng/mL, about 1350 h·ng/mL, about 1400 h·ng/mL, about 1450 h·ng/mL, about 1500 h·ng/mL, or about 1550 h·ng/mL, inclusive of all values and subranges therebetween.

In other embodiments, the solid dosage forms described herein provide an AUC₍₀₋₂₄₎ of within the range of about 80% to about 125% of about 1305 h·ng/mL to about 1763 h·ng/mL of amphotericin B (i.e., 1534±229 h·ng/mL) when orally administered to a male Sprague Dawley rat at dosage of 10 mg/kg, e.g., about 1000 h·ng/mL, about 1050 h·ng/mL, about 1100 h·ng/mL, about 1150 h·ng/mL, about 1200 h·ng/mL, about 1250 h·ng/mL, about 1300 h·ng/mL, about 1350 h·ng/mL, about 1400 h·ng/mL, about 1450 h·ng/mL, about 1500 h·ng/mL, about 1550 hang/mL, about 1600 h·ng/mL, about 1650 h·ng/mL, about 1700 h·ng/mL, about 1750 h·ng/mL, about 1800 h·ng/mL, about 1850 h·ng/mL, about 1900 h·ng/mL, about 1950 h·ng/mL, about 2000 h·ng/mL, about 2050 h·ng/mL, about 2100 h·ng/mL, about 2150 h·ng/mL, about 2200 h·ng/mL, about 2250 h·ng/mL, inclusive of all values and subranges therebetween.

Conventional liquid dosage forms provide an AUC₍₀₋₄₈₎ of 2695±433 h·ng/mL of amphotericin B when orally administered to a male Sprague Dawley rat at dosage of 10 mg/kg.

In embodiments, the amphotericin B dosage forms disclosed herein provide an AUC₍₀₋₄₈₎ within the range of about 80% to about 125% of about 2262 h·ng/mL to about 3128 h·ng/mL of amphotericin B when orally administered to a male Sprague Dawley rat at dosage of 10 mg/kg, e.g. about 1750 h·ng/mL, about 1800 h·ng/mL, about 1850 h·ng/mL, about 1900 h·ng/mL, about 1950 h·ng/mL, about 2000 h·ng/mL, about 2050 h·ng/mL, about 2100 h·ng/mL, about 2150 h·ng/mL, about 2200 h·ng/mL, about 2250 h·ng/mL, about 2300 h·ng/mL, about 2350 h·ng/mL, about 2400 h·ng/mL, 2450 h·ng/mL, about 2500 h·ng/mL, about 2550 h·ng/mL, about 2600 h·ng/mL, about 2650 h·ng/mL, about 2700 h·ng/mL, about 2750 h·ng/mL, about 2800 h·ng/mL, about 2850 h·ng/mL, about 2900 h·ng/mL, about 2950 h·ng/mL, about 3000 h·ng/mL, about 3050 h·ng/mL, about 3100 h·ng/mL, about 3150 h·ng/mL, about 3200 h·ng/mL, about 3250 h·ng/mL, about 3300 h·ng/mL, about 3350 h·ng/mL, about 3400 h·ng/mL, 3450 h·ng/mL, about 3500 h·ng/mL, about 3550 h·ng/mL, about 3600 h·ng/mL, about 3650 h·ng/mL, about 3700 h·ng/mL, about 3750 h·ng/mL, about 3800 h·ng/mL, about 3850 h·ng/mL, about 3900 h·ng/mL, about 4000 h·ng/mL, including all values and subranges therebetween.

Conventional liquid dosage forms provide Tₘₐₓ of 6.3±0.9 h of amphotericin B when orally administered to a male Sprague Dawley rat at dosage of 4.5 mg/kg, or a Tₘₐₓ of 12.5±2.7 h of amphotericin B when orally administered to a male Sprague Dawley rat at dosage of 10 mg/kg.

In embodiments, the amphotericin B dosage forms disclosed herein provide a Tₘₐₓ within the range of about 80% to about 125% of about 5.4 h to about 7.2 h of amphotericin B when orally administered to a male Sprague Dawley rat at dosage of 4.5 mg/kg, e.g. about 4.1 h, about 4.2 h, about 4.3 h, about 4.4 h, about 4.5 h, about 4.6 h, about 4.7 h, about 4.8 h, about 4.9 h, about 5.0 h, about 5.1 h, about 5.2 h, about 5.3 h, about 5.4 h, about 5.5 h, about 5.6 h, about 5.7 h, about 5.8 h, about 5.9 h, about 6.0 h, about 6.1 h, about 6.2 h, about 6.3 h, about 6.4 h, about 6.5 h, about 6.6 h, about 6.7 h, about 6.8 h, about 6.9 h, or about 7.0 h, about 7.1 h, about 7.2 h, about 7.3 h, about 7.4 h, about 7.5 h, about 7.6 h, about 7.7 h, about 7.8 h, about 7.9 h, about 8.0 h, about 8.1 h, about 8.2 h, about 8.3 h, about 8.4 h, about 8.5 h, about 8.6 h, about 8.7 h, about 8.8 h, about 8.9 h, about 9 h, about 9.1 h, about 9.2 h, about 9.3 h, about 9.4 h, or about 9.5 h, inclusive of all values and subranges therebetween.

In embodiments, the amphotericin B dosage forms disclosed herein provide a Tₘₐₓ within the range of about 80% to about 125% of about 9.8 h to about 15.2 h of amphotericin B when orally administered to a male Sprague Dawley rat at dosage of 10 mg/kg, e.g. about 7.0 h, about 7.1 h, about 7.2 h, about 7.3 h, about 7.4 h, about 7.5 h, about 7.6 h, about 7.7 h, about 7.8 h, about 7.9 h, about 8.0 h, about 8.1 h, about 8.2 h, about 8.3 h, about 8.4 h, about 8.5 h, about 8.6 h, about 8.7 h, about 8.8 h, about 8.9 h, about 9 h, about 9.1 h, about 9.2 h, about 9.3 h, about 9.4 h, about 9.5 h, about 9.6 h, about 9.7 h, about 9.8 h, about 9.9 h, about 10.0 h, about 10.1 h, about 10.2 h, about 10.3 h, about 10.4 h, about 10.5 h, about 10.6 h, about 10.7 h, about 10.8 h, about 10.9 h, about 11.0 h, about 11.1 h, about 11.2 h, about 11.3 h, about 11.4 h, about 11.5 h, about 11.6 h, about 11.7 h, about 11.8 h, about 11.9 h, about 12.0 h, about 12.1 h, about 12.2 h, about 12.3 h, about 12.4 h, about 12.5 h, about 12.6 h, about 12.7 h, about 12.8 h, about 12.9 h, about 13.0 h, about 13.1 h, about 13.2 h, about 13.3 h, about 13.4 h, about 13.5 h, about 13.6 h, about 13.7 h, about 13.8 h, about 13.9 h, about 14.0 h, about 14.1 h, about 14.2 h, about 14.3 h, about 14.4 h, about 14.5 h, about 14.6 h, about 14.7 h, about 14.8 h, about 14.9 h, about 15.0 h, about 15.1 h, about 15.2 h, about 15.3 h, about 15.4 h, about 15.5 h, about 15.6 h, about 15.7 h, about 15.8 h, about 15.9 h, about 16.0 h, about 16.1 h, about 16.2 h, about 16.3 h, about 16.4 h, about 16.5 h, about 16.6 h, about 16.7 h, about 16.8 h, about 16.9 h, about 17.0 h, about 17.1 h, about 17.2 h, about 17.3 h, about 17.4 h, about 17.5 h, about 17.6 h, about 17.7 h, about 17.8 h, about 17.9 h, about 18.0 h, about 18.1 h, about 18.2 h, about 18.3 h, about 18.4 h, about 18.5 h, about 18.6 h, about 18.7 h, about 18.8 h, about 18.9 h, about 19.0 h, about 19.1 h, about 19.2 h, about 19.3 h, about 19.4 h, about 19.5 h, inclusive of all values and subranges therein.

The solid dosage forms described herein have been administered to beagle dogs, and the average blood plasma concentrations were measured following administration. In embodiments, the solid dosage forms described herein provide a blood plasma concentration within the range of about 80% to about 125% of about 7.61 ng/mL to about 52.21 ng/mL of amphotericin B between 1 and 24 hours after oral administration of a 100 mg dose to a beagle dog, e.g. about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, inclusive of all values and subranges therebetween.

In some embodiments, the solid dosage forms described herein provide a Cₘₐₓ (in a beagle dog) in the range of about 80% to about 125% of about 39.3 ng/mL to about 53.5 ng/mL of amphotericin B (i.e., 46.4±53.5 ng/mL) after oral administration of a 100 mg dose to the beagle dog, e.g. about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, inclusive of all values and subranges therebetween. In other embodiments, the solid dosage forms described herein provide a Cₘₐₓ (in a beagle dog) in the range of about 80% to about 125% of about 45.3 ng/mL to about 59.7 ng/mL of amphotericin B (i.e., 52.5±7.2 ng/mL) after oral administration of a 100 mg dose to the beagle dog, e.g. about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, inclusive of all values and subranges therebetween.

In other embodiments, the solid dosage forms described herein provide a Tₘₐₓ (in a beagle dog) in the range of about 80% to about 125% of about 9.5 h to about 18.5 h of amphotericin B (i.e., 14.0±4.5 h) after oral administration of a 100 mg dose to the beagle dog, e.g. about 7.5 h, about 8.0 h, about 9.0 h, about 9.5 h, about 10 h, about 10.5 h, about 11 h, about 11.5 h, about 12 h, about 12.5 h, about 13 h, about 13.5 h, about 14 h, about 14.5 h, about 15 h, about 15.5 h, about 16 h, about 16.5 h, about 17 h, about 17.5 h, about 18 h, about 18.5 h, about 19 h, about 19.5 h, about 20 h, about 20.5 h, about 21 h, about 21.5 h, about 22 h, about 22.5 h, about 23 h, about 23.5 h, about 24 h, about 24.5 h, about 25 h, about 25.5 h, inclusive of all values and subranges therebetween.

In other embodiments, the solid dosage forms described herein provide a Tₘₐₓ (in a beagle dog) in the range of about 80% to about 125% of about 4.7 h to about 11.3 h of amphotericin B (i.e., 8.0±3.3 h) after oral administration of a 100 mg dose to the beagle dog, e.g. about 3.5 h, about 3.6 h, about 3.7 h, about 3.8 h, about 3.9 h, about 4.0 h, about 4.1 h, about 4.2 h, about 4.3 h, about 4.4 h, about 4.5 h, about 4.6 h, about 4.8 h, about 4.9 h, about 5.0 h, about 5.1 h, about 5.2 h, about 5.3 h, about 5.4 h, about 5.5 h, about 5.6 h, about 5.7 h, about 5.8 h, about 5.9 h, about 6.0 h, about 6.1 h, about 6.2 h, about 6.3 h, about 6.4 h, about 6.5 h, about 6.6 h, about 6.7 h, about 6.8 h, about 6.9 h, about 7.0 h, about 7.1 h, about 7.2 h, about 7.3 h, about 7.4 h, about 7.5 h, about 7.6 h, about 7.7 h, about 7.8 h, about 7.9 h, about 8.0 h, about 8.1 h, about 8.2 h, about 8.3 h, about 8.4 h, about 8.5 h, about 8.6 h, about 8.7 h, about 8.8 h, about 8.9 h, about 9 h, about 9.1 h, about 9.2 h, about 9.3 h, about 9.4 h, about 9.5 h, about 9.6 h, about 9.7 h, about 9.8 h, about 9.9 h, about 10.0 h, about 10.1 h, about 10.2 h, about 10.3 h, about 10.4 h, about 10.5 h, about 10.6 h, about 10.7 h, about 10.8 h, about 10.9 h, about 11.0 h, about 11.1 h, about 11.2 h, about 11.3 h, about 11.4 h, about 11.5 h, about 11.6 h, about 11.7 h, about 11.8 h, about 11.9 h, about 12.0 h, about 12.1 h, about 12.2 h, about 12.3 h, about 12.4 h, about 12.5 h, about 12.6 h, about 12.7 h, about 12.8 h, about 12.9 h, about 13.0 h, about 13.1 h, about 13.2 h, about 13.3 h, about 13.4 h, about 13.5 h, about 13.6 h, about 13.7 h, about 13.8 h, about 13.9 h, about 14.0 h, about 14.1 h, about 14.2 h, about 14.3 h, about 14.4 h, about 14.5 h, inclusive of all values and subranges therebetween.

In some embodiments, the solid dosage forms described herein provide an AUC_{0-Tlast} (ng*hr/mL) (in a beagle dog) in the range of about 80% to about 125% of about 1409 ng*hr/mL to about 1991 ng*hr/mL of amphotericin B (i.e., 1700±291 ng*hr/mL) after oral administration of a 100 mg dose to the beagle dog, e.g. about 1100 ng*hr/mL, about 1200 ng*hr/mL, about 1300 ng*hr/mL, about 1400 ng*hr/mL, about 1500 ng*hr/mL, about 1600 ng*hr/mL, about 1700 ng*hr/mL, about 1800 ng*hr/mL, about 1900 ng*hr/mL, about 2000 ng*hr/mL, about 2100 ng*hr/mL, about 2200 ng*hr/mL, about 2300 ng*hr/mL, about 2400 ng*hr/mL, about 2500 ng*hr/mL, inclusive of all values and subranges therebetween. In other embodiments, the solid dosage forms described herein provide an AUC_{0-Tlast} (ng*hr/mL) (in a beagle dog) in the range of about 80% to about 125% of about 1777 ng*hr/mL to about 2515 ng*hr/mL of amphotericin B (i.e., 2146±369 ng*hr/mL) after oral administration of a 100 mg dosage of to the beagle dog, e.g. about 1400 ng*hr/mL, about 1500 ng*hr/mL, about 1600 ng*hr/mL, about 1700 ng*hr/mL, about 1800 ng*hr/mL, about 1900 ng*hr/mL, about 2000 ng*hr/mL, about 2100 ng*hr/mL, about 2200 ng*hr/mL, about 2300 ng*hr/mL, about 2400 ng*hr/mL, about 2500 ng*hr/mL, about 2600 ng*hr/mL, about 2700 ng*hr/mL, about 2800 ng*hr/mL, about 2900 ng*hr/mL, about 3000 ng*hr/mL, about 3100 ng*hr/mL, about 3200 ng*hr/mL, inclusive of all values and subranges therebetween.

In some embodiments, the solid dosage forms described herein provide an MRTLast (in a beagle dog) in the range of about 80% to about 125% of about 26.1 hr to about 27.3 hr, of amphotericin B (i.e., 26.7±0.6 hr) after oral administration of a 100 mg dose to the beagle dog, e.g. about 20 hr, about 20.5 hr, about 21 hr, about 21.5 hr, about 22 hr, about 22.5 hr, about 23 hr, about 23.5 hr, about 24 hr, about 24.5 hr, about 25 hr, about 25.5 hr, about 26 hr, about 26.5 hr, about 27 hr, about 27.5 hr, about 28 hr, about 28.5 hr, about 29 hr, about 29.5 hr, about 30 hr, about 31.5 hr, about 32 hr, about 32.5 hr, about 33 hr, about 33.5 hr, about 34 hr, about 34.5 hr, and about 35 hr, inclusive of all values and subranges therebetween. In some embodiments, the solid dosage forms described herein provide an MRTLast (in a beagle dog) in the range of about 80% to about 125% of about 25.5 hr to about 29.1 hr, of amphotericin B (i.e., 27.3±1.8 hr) after oral administration of a 100 mg dosage of to the beagle dog, e.g. about 20 hr, about 20.5 hr, about 21 hr, about 21.5 hr, about 22 hr, about 22.5 hr, about 23 hr, about 23.5 hr, about 24 hr, about 24.5 hr, about 25 hr, about 25.5 hr, about 26 hr, about 26.5 hr, about 27 hr, about 27.5 hr, about 28 hr, about 28.5 hr, about 29 hr, about 29.5 hr, about 30 hr, about 31.5 hr, about 32 hr, about 32.5 hr, about 33 hr, about 33.5 hr, about 34 hr, about 34.5 hr, about 35 hr, about 35.5 hr, about 36 hr, about 36.5 hr, and about 37 hr, inclusive of all values and subranges therebetween.

In some embodiments, the AUC, Cₘₐₓ, Tₘₐₓ, and/or MRT_{Last} does not vary by more than 20% between the fed and fasted state. That is, in some embodiments, the percent difference in the fed and fasted state AUC_{0-Tlast} (ng*hr/mL) is less than or equal to 20%, e.g., less than or equal to about 20%, about 15%, about 10%, about 9%, about 8%, about 7%, about 6%, about 5%, about 4%, about 3%, about 2%, about 1%, about 0.5%, and about 0.1%, inclusive of all values therebetween. In some embodiments, the percent difference in the fed and fasted state Cₘₐₓ (ng*hr/mL) is less than or equal to 20%, e.g., less than or equal to about 20%, about 15%, about 10%, about 9%, about 8%, about 7%, about 6%, about 5%, about 4%, about 3%, about 2%, about 1%, about 0.5%, and about 0.1%, inclusive of all values therebetween.

In some embodiments, the solid dosage forms described herein provide a Cₘₐₓ (in a beagle dog) in the range of about 80% to about 125% of about 40.63 ng/mL to about 82.57 ng/mL (i.e., 61.6±20.97 ng/mL) after oral administration of a 500 mg dose to the beagle dog in the fasted state, for example about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, and about 105 ng/mL, inclusive of all values and subranges therebetween.

In some embodiments, the solid dosage forms described herein provide a Cₘₐₓ (in a beagle dog) in the range of about 80% to about 125% of about 44 ng/mL to about 88.75 ng/mL (i.e., 66.5±22.5 ng/mL) after oral administration of a 500 mg dose to the beagle dog in the fed state, for example about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, and about 105 ng/mL, inclusive of all values and subranges therebetween.

In some embodiments, the solid dosage forms described herein provide an AUC_{0-Tlast} (ng*hr/mL) (in a beagle dog) in the range of about 80% to about 125% of about 568 ng/mL to about 1682 ng*hr/mL (i.e., 1125±557 ng/mL) after oral administration of a 500 mg dose to the beagle dog in the fasted state, for example about 400 ng*hr/mL, about 500 ng*hr/mL, about 600 ng*hr/mL, about 700 ng*hr/mL, about 800 ng*hr/mL, about 900 ng*hr/mL, about 1000 ng*hr/mL, about 1100 ng*hr/mL, about 1200 ng*hr/mL, about 1300 ng*hr/mL, about 1400 ng*hr/mL, about 1500 ng*hr/mL, about 1600 ng*hr/mL, about 1700 ng*hr/mL, about 1800 ng*hr/mL, about 1900 ng*hr/mL, about 2000 ng*hr/mL, about 2100 ng*hr/mL, about 2200 ng*hr/mL, about 2300 ng*hr/mL, about 2400 ng*hr/mL, about 2500 ng*hr/mL, inclusive of all values and subranges therebetween.

In some embodiments, the solid dosage forms described herein provide an AUC_{0-Tlast} (ng*hr/mL) (in a beagle dog) in the range of about 80% to about 125% of about 657 ng/mL to about 1791 ng*hr/mL (i.e., 1224±567 ng/mL) after oral administration of a 500 mg dose to the beagle dog in the fed state, for example about 500 ng*hr/mL, about 600 ng*hr/mL, about 700 ng*hr/mL, about 800 ng*hr/mL, about 900 ng*hr/mL, about 1000 ng*hr/mL, about 1100 ng*hr/mL, about 1200 ng*hr/mL, about 1300 ng*hr/mL, about 1400 ng*hr/mL, about 1500 ng*hr/mL, about 1600 ng*hr/mL, about 1700 ng*hr/mL, about 1800 ng*hr/mL, about 1900 ng*hr/mL, about 2000 ng*hr/mL, about 2100 ng*hr/mL, about 2200 ng*hr/mL, about 2300 ng*hr/mL, about 2400 ng*hr/mL, about 2500 ng*hr/mL, inclusive of all values and subranges therebetween.

In some embodiments, the solid dosage forms described herein provide a Tₘₐₓ (in a beagle dog) in the range of about 80% to about 125% of about 5 h to about 13 h of amphotericin B (i.e., 9±4 h) after oral administration of a 500 mg dose to the beagle dog in the fed or fasted stated, e.g. about 3.9 h, about 4.0 h, about 4.1 h, about 4.2 h, about 4.3 h, about 4.4 h, about 4.5 h, about 4.6 h, about 4.8 h, about 4.9 h, about 5.0 h, about 5.1 h, about 5.2 h, about 5.3 h, about 5.4 h, about 5.5 h, about 5.6 h, about 5.7 h, about 5.8 h, about 5.9 h, about 6.0 h, about 6.1 h, about 6.2 h, about 6.3 h, about 6.4 h, about 6.5 h, about 6.6 h, about 6.7 h, about 6.8 h, about 6.9 h, about 7.0 h, about 7.1 h, about 7.2 h, about 7.3 h, about 7.4 h, about 7.5 h, about 7.6 h, about 7.7 h, about 7.8 h, about 7.9 h, about 8.0 h, about 8.1 h, about 8.2 h, about 8.3 h, about 8.4 h, about 8.5 h, about 8.6 h, about 8.7 h, about 8.8 h, about 8.9 h, about 9 h, about 9.1 h, about 9.2 h, about 9.3 h, about 9.4 h, about 9.5 h, about 9.6 h, about 9.7 h, about 9.8 h, about 9.9 h, about 10.0 h, about 10.1 h, about 10.2 h, about 10.3 h, about 10.4 h, about 10.5 h, about 10.6 h, about 10.7 h, about 10.8 h, about 10.9 h, about 11.0 h, about 11.1 h, about 11.2 h, about 11.3 h, about 11.4 h, about 11.5 h, about 11.6 h, about 11.7 h, about 11.8 h, about 11.9 h, about 12.0 h, about 12.1 h, about 12.2 h, about 12.3 h, about 12.4 h, about 12.5 h, about 12.6 h, about 12.7 h, about 12.8 h, about 12.9 h, about 13.0 h, about 13.1 h, about 13.2 h, about 13.3 h, about 13.4 h, about 13.5 h, about 13.6 h, about 13.7 h, about 13.8 h, about 13.9 h, about 14.0 h, about 14.1 h, about 14.2 h, about 14.3 h, about 14.4 h, about 14.5 h, about 14.6 h, about 14.7, about 14.8 h, about 14.9 h, about 15.0 h, about 15.1 h, about 15.2 h, about 15.3 h, about 15.4 h, about 15.5 h, about 15.6 h, about 15.7, about 15.8 h, about 15.9 h, about 16.0, about 16.1 h, about 16.2, and about 16.3, inclusive of all values and subranges therebetween.

In some embodiments, the solid dosage forms described herein provide an MRTLast (in a beagle dog) in the range of about 80% to about 125% of about 10.29 hr to about 14.69 hr, of amphotericin B (i.e., 12.49±2.2 hr) after oral administration of a 500 mg dose to the beagle dog in the fasted state, e.g. about 8.0 h, about 8.1 h, about 8.2 h, about 8.3 h, about 8.4 h, about 8.5 h, about 8.6 h, about 8.7 h, about 8.8 h, about 8.9 h, about 9 h, about 9.1 h, about 9.2 h, about 9.3 h, about 9.4 h, about 9.5 h, about 9.6 h, about 9.7 h, about 9.8 h, about 9.9 h, about 10.0 h, about 10.1 h, about 10.2 h, about 10.3 h, about 10.4 h, about 10.5 h, about 10.6 h, about 10.7 h, about 10.8 h, about 10.9 h, about 11.0 h, about 11.1 h, about 11.2 h, about 11.3 h, about 11.4 h, about 11.5 h, about 11.6 h, about 11.7 h, about 11.8 h, about 11.9 h, about 12.0 h, about 12.1 h, about 12.2 h, about 12.3 h, about 12.4 h, about 12.5 h, about 12.6 h, about 12.7 h, about 12.8 h, about 12.9 h, about 13.0 h, about 13.1 h, about 13.2 h, about 13.3 h, about 13.4 h, about 13.5 h, about 13.6 h, about 13.7 h, about 13.8 h, about 13.9 h, about 14.0 h, about 14.1 h, about 14.2 h, about 14.3 h, about 14.4 h, about 14.5 h, about 14.6 h, about 14.7, about 14.8 h, about 14.9 h, about 15.0 h, about 15.1 h, about 15.2 h, about 15.3 h, about 15.4 h, about 15.5 h, about 15.6 h, about 15.7, about 15.8 h, about 15.9 h, about 16.0 h, about 16.1 h, about 16.2 h, about 16.3, about 16.4 h, about 16.5 h, about 16.6 h, about 16.7 h, about 16.8 h, about 16.9 h, about 17 h, about 17.1 h, about 17.2 h, about 17.3 h, about 17.4 h, about 17.5 h, about 17.6 h, about 17.7 h, about 17.8 h, about 17.9 h, about 18.0 h, about 18.1 h, about 18.2 h, about 18.3 h, about 18.4 h, and about 18.5 h, inclusive of all values and subranges therebetween.

In some embodiments, the solid dosage forms described herein provide an MRTLast (in a beagle dog) in the range of about 80% to about 125% of about 10.29 hr to about 14.69 hr, of amphotericin B (i.e., 12.06±1.4 hr) after oral administration of a 500 mg dose to the beagle dog in the fed state, e.g. about 8.0 h, about 8.1 h, about 8.2 h, about 8.3 h, about 8.4 h, about 8.5 h, about 8.6 h, about 8.7 h, about 8.8 h, about 8.9 h, about 9 h, about 9.1 h, about 9.2 h, about 9.3 h, about 9.4 h, about 9.5 h, about 9.6 h, about 9.7 h, about 9.8 h, about 9.9 h, about 10.0 h, about 10.1 h, about 10.2 h, about 10.3 h, about 10.4 h, about 10.5 h, about 10.6 h, about 10.7 h, about 10.8 h, about 10.9 h, about 11.0 h, about 11.1 h, about 11.2 h, about 11.3 h, about 11.4 h, about 11.5 h, about 11.6 h, about 11.7 h, about 11.8 h, about 11.9 h, about 12.0 h, about 12.1 h, about 12.2 h, about 12.3 h, about 12.4 h, about 12.5 h, about 12.6 h, about 12.7 h, about 12.8 h, about 12.9 h, about 13.0 h, about 13.1 h, about 13.2 h, about 13.3 h, about 13.4 h, about 13.5 h, about 13.6 h, about 13.7 h, about 13.8 h, about 13.9 h, about 14.0 h, about 14.1 h, about 14.2 h, about 14.3 h, about 14.4 h, about 14.5 h, about 14.6 h, about 14.7, about 14.8 h, about 14.9 h, about 15.0 h, about 15.1 h, about 15.2 h, about 15.3 h, about 15.4 h, about 15.5 h, about 15.6 h, about 15.7, about 15.8 h, about 15.9 h, about 16.0 h, about 16.1 h, about 16.2 h, about 16.3, about 16.4 h, about 16.5 h, about 16.6 h, about 16.7 h, about 16.8 h, about 16.9 h, about 17 h, about 17.1 h, about 17.2 h, about 17.3 h, about 17.4 h, about 17.5 h, about 17.6 h, about 17.7 h, about 17.8 h, about 17.9 h, about 18.0 h, about 18.1 h, about 18.2 h, about 18.3 h, about 18.4 h, and about 18.5 h, inclusive of all values and subranges therebetween.

The amphotericin B dosage forms described may be administered according to any suitable dosing regimen which is sufficient to treat a condition in a subject in need thereof. In particular embodiments, the subject is administered an amphotericin B formulation as described herein one or more, two or more, three or more, four or more, five or more, or six or more times, with a duration of time occurring between each provision. In some embodiments, it may be necessary to administer multiple dosage forms at the same time in order to provide the required dose. In particular embodiments, the subject (e.g., a human) is provided with the amphotericin B formulation once, twice, three times, four times, five times, six times, seven times, eight times, nine times, or ten times, with a duration of time between each provision. In particular embodiments, a subject is provided with the amphotericin B formulation about once per day for about four days, about once per day for about five days, about once per day for about six days, or about once per day for about one week. In particular embodiments, a subject is provided with the amphotericin B formulation once a day, twice a day, three times a day or four times a day, e.g., for any of the durations of time described herein. In particular embodiments, the subject is provided with the amphotericin B formulation about once a day, twice a day, three times a day, four times a day, or once every two days for about three days, four days, five days six days, one week, two weeks, three weeks, one month or two months, or longer. In particular embodiments, the days and/or weeks are consecutive. In some embodiments, the amphotericin B dosage forms described herein are formulated for administration once daily.

In some embodiments, the total daily dosage of amphotericin B is an amount in the range of from about 50 mg/day to about 1500 mg/day, e.g., about 100 mg/day, about 150 mg/day, about 200 mg/day, about 250 mg/day, about 200 mg/day, about 250 mg/day, about 300 mg/day, about 350 mg/day, about 400 mg/day, about 450 mg/day, about 500 mg/day, about 550 mg/day, about 600 mg/day, about 650 mg/day, about 700 mg/day, about 750 mg/day, about 800 mg/day, about 850 mg/day, about 900 mg/day, about 950 mg/day, about 1000 mg/day, about 1050 mg/day, about 1100 mg/day, about 1150 mg/day, about 1200 mg/day, about 1250 mg/day, about 1200 mg/day, about 1250 mg/day, about 1300 mg/day, about 1350 mg/day, about 1400 mg/day, about 1450 mg/day, or about 1500 mg/day, inclusive of all values and subranges therein.

In some embodiments, a subject is provided with an amphotericin B formulation disclosed herein multiple times per day. In some such embodiments, amphotericin B is present in the single dosage in an amount in the range of from about 50 mg/day to about 1500 mg/day, e.g., about 100 mg/day, about 150 mg/day, about 200 mg/day, about 250 mg/day, about 200 mg/day, about 250 mg/day, about 300 mg/day, about 350 mg/day, about 400 mg/day, about 450 mg/day, about 500 mg/day, about 550 mg/day, about 600 mg/day, about 650 mg/day, about 700 mg/day, about 750 mg/day, about 800 mg/day, about 850 mg/day, about 900 mg/day, about 950 mg/day, about 1000 mg/day, about 1050 mg/day, about 1100 mg/day, about 1150 mg/day, about 1200 mg/day, about 1250 mg/day, about 1200 mg/day, about 1250 mg/day, about 1300 mg/day, about 1350 mg/day, about 1400 mg/day, about 1450 mg/day, or about 1500 mg/day, inclusive of all values and subranges therein.

In some embodiments, a single dose of the amphotericin B formulations disclosed herein includes multiple dosage forms (e.g., multiple capsules). For example, in some embodiments, a single dose of an amphotericin B formulation can include at least about 1 dosage form, at least about 2 dosage forms, at about least 3 dosage forms, at about least 4 dosage forms, at about least 5 dosage forms, at least about 6 dosage forms, at least about 7 dosage forms, at least about 8 dosage forms, at least about 9 dosage forms, or at least about 10 dosage forms, etc. In other embodiments, a single dose of an amphotericin B formulation include from about 1 dosage form to about 10 dosage forms, e.g., about 2, about 3, about 4, about 5, about 6, about 7, about 8, or about 9 dosage forms, inclusive of all values and subranges therein.

The amphotericin B dosage forms may be administered to treat any infection which is responsive to amphotericin B. In some embodiments, the amphotericin dosage forms described herein may be used to treat infectious diseases, such as fungal infections, human immunodeficiency virus (HIV), and parasitic infections. Infectious diseases treatable by the method and formulations disclosed herein include fungal infections (aspergillosis, blastomycosis, candidiasis, coccidioidomycosis, crytococcosis, histoplasmosis, mucormycosis, paracoccidioidomycosis, and sporotrichosis), visceral leishmaniasis, cutaneous leishmaniasis, Chagas disease, and Febrile neutropenia. Amphotericin B has been shown to bind to amyloid and prevent the formation of fibrils. Accordingly, the Amphotericin B disclosed herein may be useful for the treatment of diseases associated with fibril formations, such as Alzheimer's disease.

In some embodiments, the disclosure provides methods for treating visceral leishmaniasis comprising orally administering a solid dosage form described herein comprising an effective amount of amphotericin B to a subject in need thereof. In another embodiment, the disclosure provides for a method of treating a fungal infection comprising orally administering a solid dosage form described herein comprising an effective amount of amphotericin B described herein to a subject in need thereof. In particular embodiments, a therapeutically effective amount of amphotericin B is sufficient to achieve a blood plasma level of 0.01 μM to 10 mM, 0.01 μM to 1 mM, 0.01 μM to 100 nM, or 0.01 μM to 10 mM. The therapeutically effective amount of amphotericin B administered can vary depending on the subject and the severity of the condition. In one embodiment, the therapeutically effective amount can range from about 0.01 to about 1000 mg/kg, about 0.1 to about 100 mg/kg, about 0.5 to about 50 mg/kg, about 1 to about 20 mg/kg subject body weight, or about 5 to about 10 mg/kg, e.g., about 5, about 6, about 7, about 8, about 9, or about 10 mg/kg.

### EXAMPLES

### Example 1: Materials and Methods

Table 1 provides a description of the materials used in the analytical studies described herein. The amphotericin (AmpB) was stored at 2-8° C., protected from light and moisture. Other materials were stored at room temperature (RT).

TABLE 1
Materials

| Material (Trade Name) | Functionality | Lot No. | Supplier |
|---|---|---|---|
| Amphotericin B | API | C00729 | Xellia |
| | | | Pharmaceuticals |
| Ethanol | Solvent | E00400 | Commercial Alcohol |
| Lauroyl polyoxylglycerides (Gelucire ® 44/14) | Filler | 136981 | Gattefossé |
| Glyceryl monooleate type 40 (Peceol ®) | | 147329 | Gattefossé |
| Polyethylene glycol succinate (Vitamin E | | 1301080017 | Isochem |
| TPGS) | | | |
| Microcrystalline cellulose type 101 | | C00465 | Blanver |
| (Tabulose ® 101) | | | |
| Mannitol (Pearlitol ® 160C) | | C00464 | Roquette |
| Silicified microcrystalline cellulose | | C00618 | JRS Pharma |
| (Prosolv ® HD 90) | | | |
| Polyvinyl pyrrolidone (Plasdone ® K-29/32) | Binder | C00605 | Ashland |
| Croscarmellose sodium type A (Solutab ®) | Disintegrant | C00593 | Blanver |
| Colloidal silicon dioxide (Aerosil ® 200) | Glidant | C00449 | Degussa |
| Magnesium stearate (Ligamed ® MF-2-V) | Lubricant | C00124 | Peter Greven |
| Hard gelatin capsule size 0 | — | 70965701 | Capsugel |
| Hard gelatin capsule size 00 | — | C00420 | Capsugel |

Sample Preparation 1
Empty the contents of 2 capsules into a 200 ml low actinic flask.
Add NMP (˜80% of the volume) and sonicate for 15 minutes, with ice pack in the bath.
Shake for 15 minutes.
Mix and allow solution to equilibrate to room temperature.
Dilute to volume with NMP.
Dilute 5 mL of the above solution to 50 mL with Diluent A (25% ammonium acetate solution/25% NMP/50% methanol).
Filter with a 0.45 μm nylon filter, discarding the first 3 ml.

Sample Preparation 2
Empty the contents of 2 capsules into a 200 ml low actinic flask.
Add 50 ml of NMP and sonicate for 15 minutes, with ice pack in the bath.
Shake for 15 minutes.
Add ˜90% of the volume of Diluent B (ammonium acetate solution/methanol 1:2).
Mix well and allow solution to equilibrate to room temperature.
Dilute to volume with Diluent B.
Dilute 5 mL of the above solution to 50 mL with Diluent A (25% ammonium acetate solution/25% NMP/50% methanol).
Filter with a 0.45 μm nylon filter, discarding the first 3 ml.

Sample Preparation 3
Transfer the contents and empty gelatin capsules of 4 capsules into a 500 ml low actinic flask.
Add 125 ml of NMP and sonicate for 30-45 minutes (with ice packs in the bath to minimize heating) until the sample is completely dispersed. Shake vigorously at regular intervals during sonication. Note: The capsule shells remain intact.
Add ˜90% of the volume of Diluent B (ammonium acetate solution/methanol 1:2).
Mix well and allow solution to equilibrate to room temperature (placed in refrigerator to cool quickly).
Dilute to volume with Diluent B.
Dilute 3 mL of the above solution to 25 mL with Diluent A (25% ammonium acetate solution/25% NMP/50% methanol).
Filter with a 0.45 μm nylon filter, discarding the first 3 ml.

Sample Preparation 4
Transfer the contents of 3 capsules into a 500 ml low actinic flask.
Complete to volume with 0.5% SDS in water.
Add a stir bar and stir for a least 90 minutes.
Dilute 8 mL of the above solution to 50 mL with 0.5% SDS in water
Filter with a 0.45 μm nylon filter, discarding the first 3 ml.

### Example 2: Solid Formulations

Amphotericin B with Gelucire/Peceol/TPGS containing formulations were prepared as shown in Tables 2-4 based on the reference iCo/Wasan liquid formulation in Table 5.

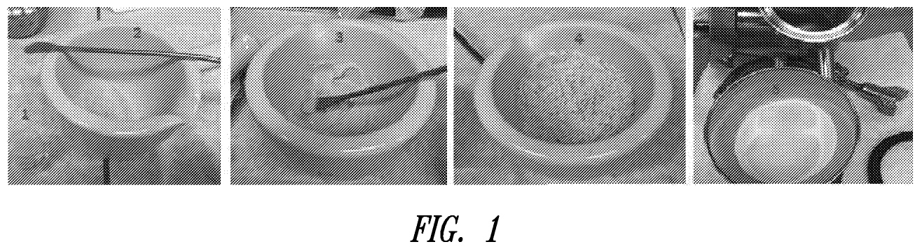

First Gelucire and TPGS were melted and weighed, both in a same container. Peceol was weighed and added to the Gelucire-TPGS mixture. Ethanol was weighed and added to the Gelucire-TPGS-Peceol mixture and mixed using a stirring heating plate at a temperature of about 40° C. until all ingredients dissolved (#1 in FIG. 1). The solution was added to API (#2 in FIG. 1) and mixed for about 5 min using a pestle. This mixture was ‘creamy’ at 25° C. (#3 in FIG. 1). The Internal phase powder excipients were mixed separately using a V-blender at 25 rpm for 2 min. Both mixtures were mixed using a pestle/mortar for about 5 min. The resulting mixture (#4 in FIG. 1) was placed in an oven at 40° C. for 1-2 h to evaporate ethanol and then removed from oven and kept for about 2 h at 22-25° C. The granules were obtained by milling through a 20 mesh (850 μm) screen. The lubricant (e.g., magnesium stearate) was mixed with granules using a V-blender for 2 min. The final blend (#5 in FIG. 1) was encapsulated into size “0” hard shell gelatin capsules (435 mg/caps). The capsules were filled by volume using tapping/tamping technique.

TABLE 2
Formulation 1

| Item | Ingredient | % w/w | mg/unit | g/batch |
|---|---|---|---|---|
| a | Amphotericin B | 23.0 | 100 | 4.60 |
| b | Mannitol 160C | 34.5 | 150 | 6.90 |
| c | Tabulose 101 | 34.3 | 149 | 6.85 |
| d | Colloidal silicon dioxide | 2.3 | 10 | 0.46 |
| e | TPGS | 0.2 | 1 | 0.05 |
| f | Peceol | 2.3 | 10 | 0.46 |
| g | Gelucire 44/14 | 2.3 | 10 | 0.46 |
| h | Ethanol 100% (evaporated | (30.0) | — | (6.00) |
| | during the process) | | | |
| i | Magnesium stearate | 1.1 | 5 | 0.23 |

| | | | |
|---|---|---|---|
| Total: | 100 | 435 | 20 |

Items a-h are internal phase components, and item i is the external phase component.

TABLE 3
Formulation 2

| Item | Ingredient | % w/w | mg/unit | g/batch |
|---|---|---|---|---|
| a | Amphotericin B | 23.0 | 100 | 4.60 |
| b | Prosolv HD90 | 66.0 | 287 | 13.21 |
| c | Croscarmellose sodium | 5.0 | 22 | 1.00 |
| d | TPGS | 0.2 | 1 | 0.05 |
| e | Peceol | 2.3 | 10 | 0.46 |
| f | Gelucire 44/14 | 2.3 | 10 | 0.46 |
| g | Ethanol 100% (evaporated | (30.0) | — | (6.00) |
| | during the process) | | | |
| h | Magnesium stearate | 1.1 | 5 | 0.23 |

| | | | |
|---|---|---|---|
| Total: | 100 | 435 | 20 |

Items a-g are internal phase components, and item h is the external phase component.

TABLE 4
Formulation 3

| Item | Ingredient | % w/w | mg/unit | g/batch |
|---|---|---|---|---|
| a | Amphotericin B | 23.0 | 100 | 4.60 |
| b | Tabulose 101 | 66.0 | 287 | 13.21 |
| c | Plasdone K-29/32 | 5.0 | 22 | 1.00 |
| d | TPGS | 0.2 | 1 | 0.05 |
| e | Peceol | 2.3 | 10 | 0.46 |
| f | Gelucire 44/14 | 2.3 | 10 | 0.46 |
| g | Ethanol 100% (evaporated | (30.0) | — | (6.00) |
| | during the process) | | | |
| h | Magnesium stearate | 1.1 | 5 | 0.23 |

| | | | |
|---|---|---|---|
| Total: | 100 | 435 | 20 |

Items a-g are internal phase components, and item h is the external phase component.

TABLE 5
iCo/Wasan Formulation compared to Formulations 1-3

| | iCo/Wasan | | Formulation 1 | | Formulation 2 | | Formulation 3 | |
|---|---|---|---|---|---|---|---|---|
| Ingredient | Formulation | | (L268-01016) | | (L268-01017) | | (L268-01018) | |
| Amphotericin B | 150 | mg | 100 | mg | 100 | mg | 100 | mg |
| TPGS | 1.5 | mL | 1 | mg | 1 | mg | 1 | mg |
| Peceol | 14.25 | mL | 10 | mg | 10 | mg | 10 | mg |
| Gelucire 44/14 | 14.25 | mL | 10 | mg | 10 | mg | 10 | mg |
| Ethanol* 100% | (40 | mL) | (6 | g) | (6 | g) | (6 | g) |

| Mannitol 160C | — | 150 | mg | — | | — | |
|---|---|---|---|---|---|---|---|
| Tabulose 101 | — | 149 | mg | — | | 287 | mg |
| Aerosil 200 | — | 10 | mg | — | | — | |
| Prosolv | — | — | | 287 | mg | — | |
| Croscarmellose sodium | — | — | | 22 | mg | — | |
| Plasdone K-29/32 | — | — | | — | | 22 | mg |
| Magnesium stearate | — | 5 | mg | 5 | mg | 5 | mg |

### Example 2. Scale-Up of Solid Formulations from Example 1

Formulation 1 and Formulation 2 were scaled-up from 20 to 100 g (Tables 6-7; Formulation 1A and Formulation 2A, respectively). The granulation was done using a GMX top-drive high-shear granulation/mixing system where the Gelucire, Peceol and TPGS were dissolved in ethanol and this solution was added at 26 g/min and mixed to API at 60 rpm for 3 min. Powdered ingredients were separately mixed using a V-blender for 2 min. This powder blend was added to Gelucire/Peceol/TPGS/Ethanol/drug mixture and mixed for 6 min at impeller/chopper speeds 850/1800 rpm. The ethanol was then removed using a fluid bed dryer at 40° C. The fluidization was maintained until volatile compounds content was less or equal 3% (about 20 min). The volatile compounds content was determined by loss on drying (LOD) technique. The dried granules were sized by screening through an 18 mesh sieve followed by final lubrication.

TABLE 6
Formulation 1A

| Item | Ingredient | % w/w | mg/unit | g/batch |
|---|---|---|---|---|
| a | Amphotericin B | 23.0 | 100 | 23.0 |
| b | Mannitol 160C | 34.5 | 150 | 34.5 |
| c | Tabulose 101 | 34.3 | 149 | 34.3 |
| d | Colloidal silicon dioxide | 2.3 | 10 | 2.3 |
| e | TPGS | 0.2 | 1 | 0.2 |
| f | Peceol | 2.3 | 10 | 2.3 |
| g | Gelucire 44/14 | 2.3 | 10 | 2.3 |
| h | Ethanol 100% (evaporated | (30.0) | — | (30.0) |
| | during the process) | | | |
| i | Magnesium stearate | 1.1 | 5 | 1.1 |

| | | | |
|---|---|---|---|
| Total: | 100 | 435 | 100 |

Items a-h are internal phase components, and item i is the external phase component.

TABLE 7
Formulation 2A

| Item | Ingredient | % w/w | mg/unit | g/batch |
|---|---|---|---|---|
| a | Amphotericin B | 23.0 | 100 | 23.0 |
| b | Prosolv HD90 | 66.0 | 287 | 66.0 |
| c | Croscarmellose sodium | 5.0 | 22 | 5.0 |
| d | TPGS | 0.2 | 1 | 0.2 |
| e | Peceol | 2.3 | 10 | 2.3 |
| f | Gelucire 44/14 | 2.3 | 10 | 2.3 |
| g | Ethanol 100% (evaporated | (30.0) | — | (30.0) |
| | during the process) | | | |
| h | Magnesium stearate | 1.1 | 5 | 1.1 |

| | | | |
|---|---|---|---|
| Total: | 100 | 435 | 100 |

Items a-g are internal phase components, and item h is the external phase component.

### Example 3. Semi-Solid Formulations

Semi-solid lipid based formulations (Formulation 4 and Formulation 5, Table 8A) filled into capsules for oral administration were prepared as per iCo formula composition (Table 8). However, in contrast to the iCo/Wasan liquid approach, a melt method was used. Indeed, the semi-solids excipients (Gelucire/TPGS) were melted, weighed and mixed with Peceol (liquid excipient) using a hot-plate magnetic-stirrer at 35-40° C. until a clear solution was obtained. The heating was stopped and the AmpB was added and mixed for 5 min. The liquid final blend was maintained under agitation and hot filled into size 00 hard gelatin capsules (fill weight: 804 mg) containing 4 mg AmpB. An additional lot was manufactured (Formulation 5) that contains the same amount of lipid excipients but more AmpB was ‘spiked’ to produce 100 mg dose capsules (fill weight: 900 mg).

TABLE 8A
iCo/Wasan Liquid Formulation, Formulation 4 and Formulation 5

| | iCo/Wasan Formulation | | | Formuation 4 | | Formuation 5 | |
|---|---|---|---|---|---|---|---|
| Ingredient | qty/dose | | % w/w | mg/dose | % w/w | mg/dose | % w/w |
| Amphotericin B | 150 | mg | 0.5 | 4 | 0.5 | 100 | 11.1 |
| TPGS | 1.5 | mL | 5 | 40 | 5 | 40 | 4.4 |
| Peceol | 14.25 | mL | 47.3 | 380 | 47.3 | 380 | 42.2 |
| Gelucire 44/14 | 14.25 | mL | 47.3 | 380 | 47.3 | 380 | 42.2 |
| Total: | 30 | mL | 100 | 804* | 100 | 900* | 100 |

*= 0.95 mL

### Example 4. Scale up of Semi-Solid Formulation from Example 3

The lipid based Formulation 5 was scaled-up from 23 to 360 g batch size (Table 8B). Each excipient was melted in its original container, followed by stirring to ensure homogeneity before sampling. The weighed molten samples were mixed together and AmpB was added under agitation. The mixture was maintained at 40° C. and under constant agitation for at least 30 minutes to ensure complete dispersion/solubilization. The final mixture was filled into hard gelatin capsules. Once the capsules' content cooldown to room temperature, the capsules were sealed using a mixture of purified water and Ethanol (50:50 v/v). A few droplets of solution were gently applied around the junction of the closed capsules' body and cap. Exceeding solution was immediately wiped out using a clean and dry cloth. The capsules were allowed to dry individually by resting vertically on a Cooper plate. Sealed capsules were stored at 4° C. until the start of the stability study.

TABLE 8B
100 mg Amphotericin B Lipid Formulation 5A

| Ingredient | mg/dose | % w/w | g/batch |
|---|---|---|---|
| Amphotericin B | 100 | 11.1 | 40.0 |
| TPGS | 40 | 4.4 | 16.0 |
| Peceol | 380 | 42.2 | 152.0 |
| Gelucire 44/14 | 380 | 42.2 | 152.0 |
| Total: | 900 | 100 | 360 |

### Example 5. Physical and Chemical Characterization

Final blend was evaluated by bulk/tapped density and powder flow properties and residual solvents.

Bulk/Tapped Density—Powder Flow Properties—USP<616>

Bulk and tapped densities were determined using the USP <616> method with a Vanderkamp tap density tester model 10700 (VanKel Industries) and a Mettler Toledo balance model AT200. Each parameter was determined in duplicate using a 50 mL graduated glass cylinder. The bulk density was determined by measuring the volume of a known mass of powder sample in a graduated cylinder while the tapped density was measured by mechanically tapping the measuring cylinder until no further volume change was observed. The powder flow properties were evaluated using the Carr's Compressibility Index and Hausner ratio as described in the next paragraphs.

Carr's Compressibility Index (CI):

This flow property was calculated using bulk and tapped density data when fitted into the following equation: Compressibility Index=(Tapped density −Bulk density)/Tapped density×100%

Hausner Ratio (H):

This flow property was calculated as the ratio of tapped to bulk density.

The Compressibility Index (CI) and Hausner ratio (H) values interpretation as per USP <1174> as well as descriptive qualitative examples are presented in Table 9.

TABLE 9
Scale of Flowability

| Compressibility | Flow | Hausner | |
|---|---|---|---|
| Index (%) | Character | Ratio | Examples |
| <10 | Excellent | 1.00-1.11 | Free-flowing granules |
| 11-15 | Good | 1.12-1.18 | Powdered granules |
| 16-20 | Fair | 1.19-1.25 | Coarse powders |
| 21-25 | Passable | 1.26-1.34 | Fine powders |
| 26-31 | Poor | 1.35-1.45 | Fluidizable powders |
| 32-37 | Very poor | 1.46-1.59 | Cohesive powders |
| >38 | Very, very poor | >1.60 | Very cohesive powders |

Densities and powder flow properties are shown in Table 10. The formulations exhibit sufficient flowability. To fill 435 mg of final blend from Formulation 1 into size 0 capsules tapping and tamping was required. Bulk density could be increased by high shear granulation, using denser grades of excipients, e.g. microcrystalline cellulose type 200 or 302, or high functionality and multifunctional excipients such silicified microcrystalline cellulose (combination of microcrystalline cellulose and colloidal silicon dioxide). Silicified microcrystalline cellulose (Prosolv HD 90) has a bulk density 0.38-0.50 g/cm³ and was used in Formulation 2 resulting in an increased bulk density.

TABLE 10
Density and Flow Properties (n = 2)

| | | | Flow | | |
|---|---|---|---|---|---|
| | Density | | Properties | | |
| | Parameters | | Carr's | | |
| | Bulk | Tapped | Index | Hausner | Flow- |
| Lot No. | (g/cm³) | (g/cm³) | (%) | Ratio | ability |
| Formulation 1 | 0.40/0.41 | 0.49/0.50 | 18/18 | 1.22/1.22 | Fair |
| Formulation 2 | 0.46/0.45 | 0.54/0.53 | 15/14 | 1.18/1.17 | Good |
| Formulation 3 | 0.33/0.33 | 0.38/0.37 | 14/13 | 1.16/1.14 | Good |

### Capsule Weight Uniformity

As an example to show adequate flow properties, 12 capsules of Formulation 1 were tested for statistics (Table 11). Weight uniformity was confirmed with RSD <6.0% and no unit is outside the range of 85-115% of label claim.

TABLE 11
Encapsulation Statistics for 100 mg Amphotericin
B in Size 0 Capsules (n = 12) for Formulation 1.

| | Value |
|---|---|
| Statistic | (mg, Total weight) |
| Average | 527.8 |
| Stdev | 1.8 |
| RSD (%) | 0.3 |
| Min | 525.0 |
| Max | 529.9 |

The statistics for the semi-solid formulations are shown in Table 12. Formulation 4 capsules were filled by weight to approximately 90% of the capsule's volume to obtain 4 mg Amphotericin B/caps. Formulation 5 and Formulation 5A with 100 mg Amphotericin B/caps were filled to 100% of the capsule's volume.

TABLE 9
Encapsulation Statistics for Amphotericin B Semi-Solids
Formulations in Size 00 Capsules (n = 6) for
Formulation 4, Formulation 5, and Formulation 5A.

| | Formulation 4 | Formulation 5 | Formulation 5A |
|---|---|---|---|
| | (4 mg Amp. B/ | (100 mg Amp. B/ | (100 mg Amp. B/ |
| Statistics | cap) | caps) | caps) |
| Average Total | 919.1 | 1014.8 | 1011.9 |
| Wt (mg) | | | |
| Stdev | 0.9 | 6.9 | 7.5 |
| RSD (%) | 0.1 | 0.7 | 0.7 |
| Min | 918.1 | 1003.5 | 1003.8 |
| Max | 920.4 | 1022.1 | 1027.1 |

### Residual Solvents

Determination of residual solvents was carried out by thermal gravimetric analysis (TGA) using a TA Instrument Q50 thermogravimetric analyzer at scanning speed of 10° C. min′ over a temperature range of 25 to 200° C. The samples (11-13 mg) were heated in a platinum open pan in nitrogen atmosphere (60 mL min⁻¹).

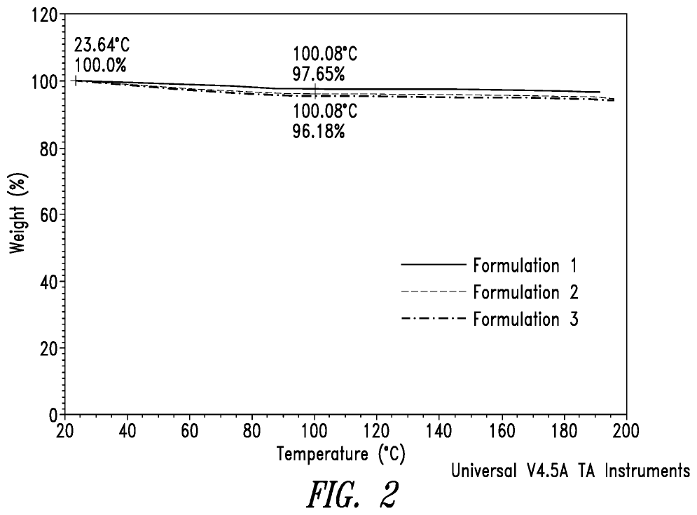

TGA curves of Amphotericin B (23%) solid oral dose formulations' final blends are illustrated in FIG. 2. TGA show weight loss of 2.4-3.8% between 25 and 100° C. which is typically associated with evaporation of volatiles compounds (solvents and moisture). This weight loss is low considering that the moisture content of microcrystalline cellulose is between 3 and 5% (moisture data from CofA). As a consequence, for samples containing higher quantity of microcrystalline cellulose such as Formulation 2 and Formulation 3, it is normal that the weight loss appeared slightly increased (3.8%) when compared with Formulation 1 (2.4%).

### Example 6. Analytical Testing

Formulation 1

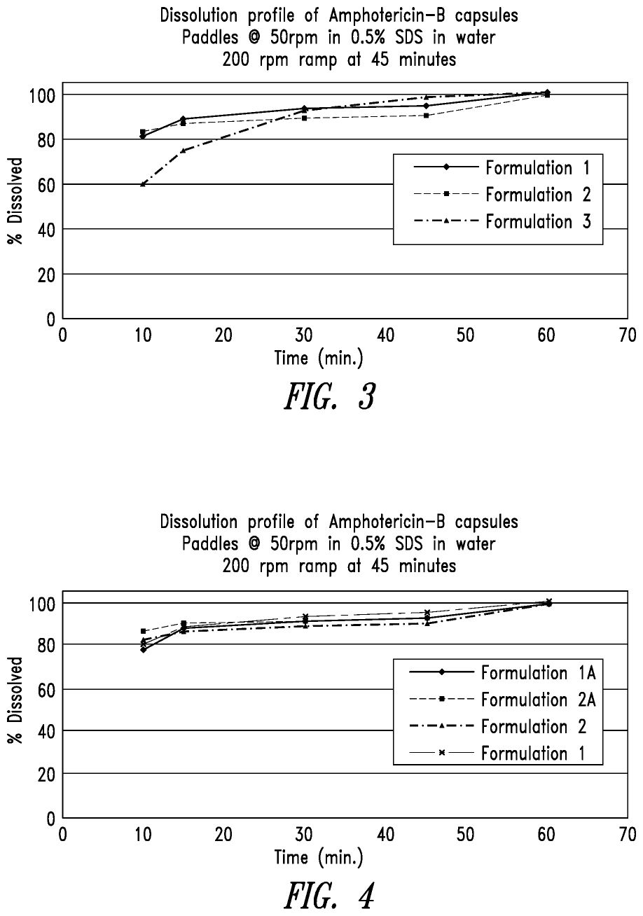

The assay and related substances results for Amphotericin B formulations are shown in Table 13. Replicate 1 and 2 were prepared using Sample Preparation 1 and Replicate 3 was prepared using Sample Preparation 2, in an attempt to minimise the consumption of NMP in the diluent. Similar extraction efficiency was obtained with both sample preparation procedures. The extraction procedure for the assay and related substances achieved ˜95% recovery. The dissolution profile is fairly rapid with 95% released at 45 minutes (FIG. 3). After an increase in paddle speed, 100% released was achieved.

Formulation 2 and Formulation 3

The assay and related substances results for Amphotericin B carried out using a modified extraction procedure are shown in Table 14. The contents and emptied capsule shells from 4 capsules were extracted per sample replicate using Sample Preparation 3 (see Method for details). Some samples required much longer sonication time to break up the agglomerates formed. The longer sonication time also increased the amount of degradation produced during sample preparation. The dissolution profiles are shown in FIG. 3. Formulation 3 appears somewhat slower initially but rapidly rejoins the profiles of the other formulations.

Table 15 shows the Impurity profile of AmpB used in the formulations which indicates that the process used to produce the dosage forms did not affect adversely the AmpB.

TABLE 10
Impurity Profile of AMpB

| | |
|---|---|
| Formuation 1 | Formulations 2 and 3 |

| RRT | % area | RRT | % area |
|---|---|---|---|
| 0.29 | 0.88 | 0.30 | 0.94 |
| 0.48 | 0.11 | 0.49 | 0.11 |
| 0.62 | 0.63 | 0.62 | 0.66 |
| 0.69 | 0.12 | 0.69 | 0.13 |
| 0.73 | 0.33 | 0.73 | 0.61 |
| 0.75 | 0.34 | 0.75 | ND |
| 0.78 | 0.26 | 0.78 | 0.20 |
| 0.82 | 0.65 | 0.82 | 0.60 |
| 0.88 | ND | 0.88 | 0.29 |
| 0.91 | 0.12 | 0.91 | ND |
| 1.08 | ND | 1.08 | 0.10 |
| 1.22 | ND | 1.22 | 0.66 |
| 1.25 | 0.72 | 1.25 | ND |
| 1.27 | ND | 1.27 | 0.73 |
| 1.29 | 0.60 | 1.29 | ND |
| 1.35 | 0.11 | 1.32 | 0.13 |
| 1.39 | 0.15 | 1.39 | ND |
| 1.79 | 1.06 | 1.79 | ND |
| 1.82 | ND | 1.82 | 1.05 |
| 1.91 | 0.14 | 1.96 | 0.13 |
| 2.32 | 1.43 | 2.35 | 1.38 |
| Total | 7.7 | Total | 7.7 |

Peaks ≥0.10% reported

Formulation 1A and Formulation 2A

The assay and related substances for Amphotericin-B in capsule was carried out at the initial time point (T=0) using a modified extraction procedure (Table 16). The contents from 3 capsules were extracted per sample replicate using Sample Preparation 4 (see Methods for details). The dissolution profiles for the scale-up lots are comparable to the previous lots (FIG. 4).

Formulation 4 and Formulation 5

Semi-solid lipid based formulations in capsules for oral administration were prepared as per iCo formula composition and Corealis modified process. The capsules were analysed for dissolution profiles in the current 0.1N HCl+0.5% SDS medium and in Simulated Fed Intestinal Fluid (FeSSIF pH 5.8) (Table 17, FIG. 5).

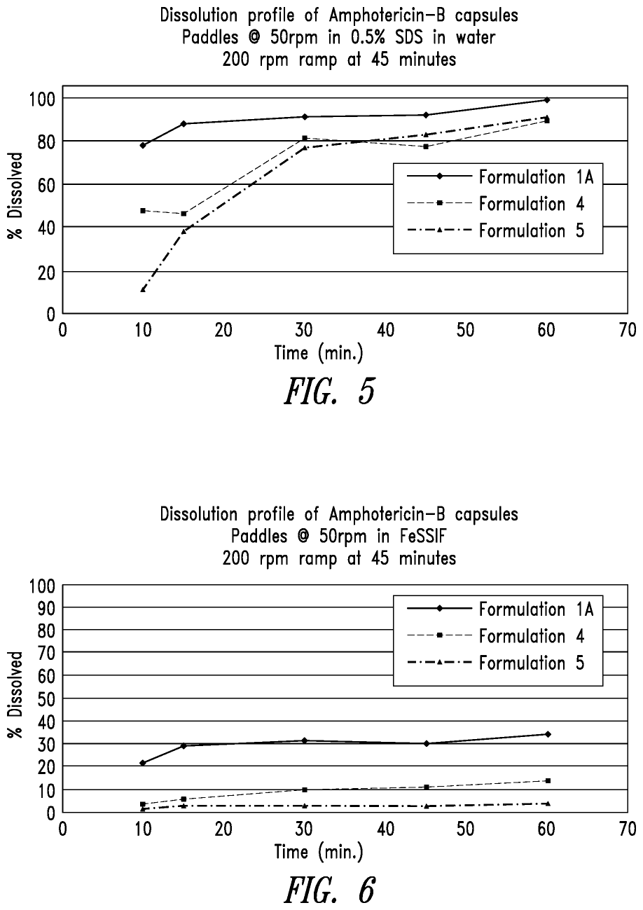

The semi-solid formulations Formulation 4 (0.5% Drug Load) and Formulation 5 (11.1% Drug Load) showed slightly slower dissolution profiles in 0.5% SDS in water up to 30 minutes when compared the ‘solid’ capsule Formulation 1A (23% Drug Load). In the FeSSIF pH 5.8 medium where Amphotericin B may be solubility limited, the dissolution profiles reached a maximum of −35% dissolved for the ‘solid’ capsule formulation and less than 15% dissolved for the semi-solid formulations (FIG. 6). In this in vitro model, the semi-solid formulations with the increased lipid concentration do not show improved dissolution profile. Both, Formulation 4 and Formulation 5 showed similar end results when compared to the solid oral dosage form Formulation 1A.

Formulation 5 and Formulation 5A

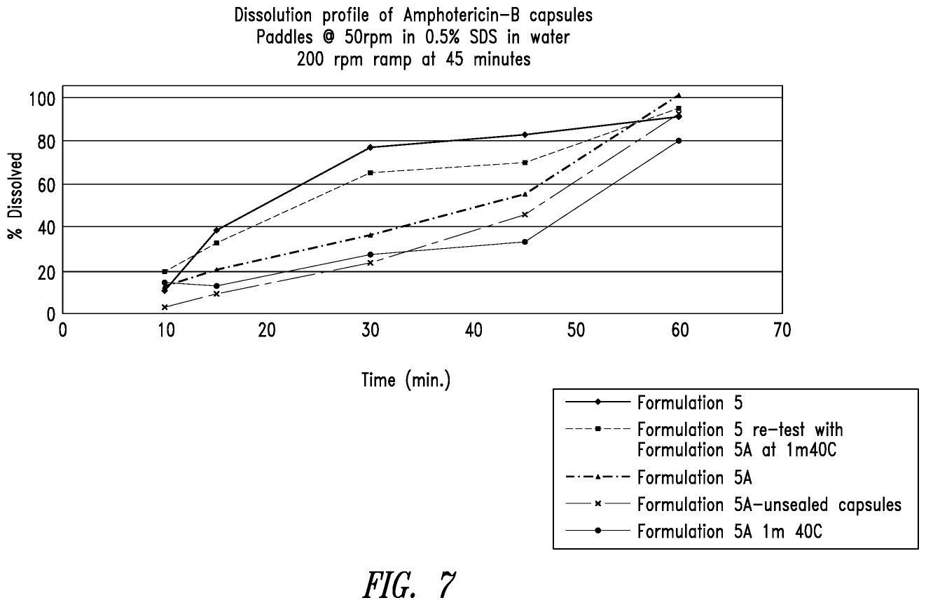

Formulation 5 and Formulation 5A are the same composition but prepared at different scale. The mixing time was increased consequently. Moreover, Formulation 5A and Formulation 5A-1 capsules comes from the same final blend with only one difference whereby Formulation 5A capsules were sealed and Formulation 5A-1 were filled later and not sealed. The initial/T=0 data shown in FIG. 7, revealed that the dissolution profiles were different for all three lots. However, after 60 minutes 90-100% of AmpB was dissolved. Subsequently, it was also discovered that lower dissolution profiles were observed for Formulation 5A stored for 1 month at 40° C./75% RH as well as for Formulation 5 stored at 5° C. for about 5 months.

Without being bound by theory, the decrease of dissolution profile as a function of time could be ascribed to different degrees of solubilizing of the AmpB during the processing of the different batch size lots.

### Example 7. Stability Study

Formulation 1A and Formulation 2A

A stability study was initiated for Formulation 1A and Formulation 2A. The capsules were packaged in 30 cc HDPE bottles with induction sealed PP caps and the bottles were stored under ICH stability conditions in humidity chambers at 25° C./60% RH and under accelerated conditions, 40° C./75% RH. The capsules were stored at 4-8° C. directly after preparation until they were placed into the stability chambers.

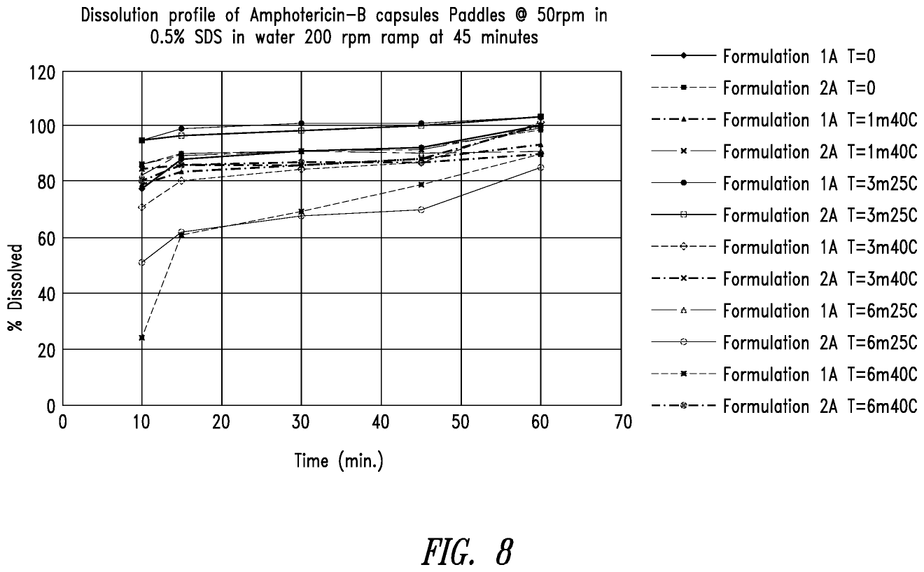

Stability testing results for 100 mg Amphotericin B capsules Formulation 1A and Formulation 2A are summarized in Tables 18 to 20. Dissolution profiles were compared in FIG. 8. Both formulations are stable for up to 6 months at 25° C./60% RH and 40° C./75% RH with no significant changes in assay, related substances, and dissolution profile when compared to initial (T=0) results.

TABLE 13
Stability Testing Results for Formulation 1A and Formulation 2A

| Sample | | Formulation 1A | Formulation 2A |
|---|---|---|---|
| Dose strength | | 100 mg/capsule | |
| Appearance | T = 0 | Yellow powder in white capsule (slight agglomerations) | |
| | T = 1 month 40° C./75% RH | Yellow powder in white capsule (slight agglomerations) | |
| | T = 3 months 25° C./60% RH | Yellow powder in white capsule (slight agglomerations) | |
| | T = 3 months 40° C./75% RH | Yellow powder in white capsule (slight agglomerations) | |
| | T = 6 months 25° C./60% RH | Yellow powder in white capsule (slight agglomerations) | |
| | T = 6 months 40° C./75% RH | Yellow powder in white capsule (slight agglomerations) | |
| Water content | T = 0 | 2.9% | 3.8% |
| | T = 1 month 40° C./75% RH | 3.2% | 4.1% |
| | T = 3 months 25° C./60% RH | 3.1% | 4.0% |
| | T = 3 months 40° C./75% RH | 3.2% | 4.4% |
| | T = 6 months 25° C./60% RH | 2.8% | 3.6% |
| | T = 6 months 40° C./75% RH | 3.1% | 4.1% |
| Assay (% LC) | T = 0 | 98.6% | 101.5% |
| Corealis -26801-AD-01 | | (n = 2: 100.6, 96.5) | (n = 2: 101.5, 101.6) |
| Rev R&D 04 | T = 1 month 40° C./75% RH | 98.9% | 101.6% |
| (T = 0 to T = 3 m) | | (n = 2: 100.4, 97.3) | (n = 2: 102.8, 100.4) |
| Corealis -26801-AD-01 | T = 3 months 25° C./60% RH | 100.0% | 97.8% |
| Rev R&D 09 | | (n = 2: 100.2, 99.7) | (n = 2: 99.1, 96.5) |
| (T = 6 m) | T = 3 months 40° C./75% RH | 97.2% | 99.3% |
| | | (n = 2: 95.7, 98.6) | (n = 2: 100.5, 98.0) |
| | T = 6 months 25° C./60% RH | 96.9% | 93.0% |
| | | (n = 5: 102.0, 96.3, 100.8, 90.3, 95.2) | (n = 5: 94.7, 95.3,95.2, 89.4, 90.6) |
| | T = 6 months 40° C./75% RH | 93.5% | 91.8% |
| | | (n = 5: 96.5, 90.0, 94.4, 92.7, 94.2) | (n = 5: 89.3, 89.6, 93.0, 97.1, 89.8) |

Note:
The assay is quantitated against the API only.
Chromatographic impurities are not taken into account.

TABLE 20
Stability Testing Results (Dissolution) for Formulation 1A and Formulation 2A

| | | Sample | | | | | |
|---|---|---|---|---|---|---|---|
| | | Formulation 1A | | | Formulation 2A | | |
| | | Formulation | | | | | |
| | | | | | Amphotericin B with Gelucire | | |
| | | Amphotericin B with Gelucire | | | 44/14-Peceol-TPGS (10 mg- | | |
| | | 44/14-Peceol-TPGS (10 mg- | | | 10 mg-1 mg), silicified | | |
| | | 10 mg-1 mg), | | | microcrystalline | | |
| | | mannitol/microcrystalline | | | cellulose/croscarmellose oral | | |
| | | cellulose oral dose formulation in | | | dose formulation in hard shell | | |
| | | hard shell capsule | | | capsule | | |
| | | Dose strength | | | | | |
| | | 100 mg/capsule | | | | | |
| | | Time | % | | Time | % | |
| | | (min.) | dissolved | Range | (min.) | dissolved | Range |
| Dissolution | T = 0 | 10 | 78 | (69-84) | 10 | 86 | (81-95) |
| 900 ml 0.5% SDS | | 15 | 88 | (77-96) | 15 | 90 | (86-97) |
| in Water | | 30 | 91 | (82-100) | 30 | 91 | (88-97) |
| paddles at 50 | | 45 | 92 | (83-100) | 45 | 92 | (88-98) |
| rpm | | 60 | 99 | (93-102) | 60 | 99 | (98-99) |
| % dissolved | | (ramp) | | | (ramp) | | |
| Corealis -26801- | T = 1 month | 10 | 80 | (74-84) | 10 | 86 | (80-95) |
| B-01 | 40° C./75% RH | 15 | 84 | (80-88) | 15 | 89 | (83-98) |
| Rev R&D 02 | | 30 | 86 | (83-90) | 30 | 91 | (85-100) |
| (n = 3) | | 45 | 88 | (85-91) | 45 | 91 | (86-101) |
| | | 60 | 93 | (89-95) | 60 | 100 | (96-106) |
| | | (ramp) | | | (ramp) | | |
| | T = 3 months | 10 | 95 | (87-109) | 10 | 95 | (92-99) |
| | 25° C./60% RH | 15 | 99 | (91-113) | 15 | 97 | (93-102) |
| | | 30 | 101 | (93-113) | 30 | 98 | (94-104) |
| | | 45 | 101 | (93-112) | 45 | 100 | (95-105) |
| | | 60 | 103 | (95-113) | 60 | 103 | (99-109) |
| | | (ramp) | | | (ramp) | | |
| | T = 3 months | 10 | 71 | (64-82) | 10 | 84 | (78-87) |
| | 40° C./75% RH | 15 | 80 | (73-86) | 15 | 86 | (81-90) |
| | | 30 | 85 | (74-93) | 30 | 86 | (81-91) |
| | | 45 | 87 | (77-94) | 45 | 88 | (82-91) |
| | | 60 | 101 | (100-103) | 60 | 100 | (96-109) |
| | | (ramp) | | | (ramp) | | |
| | T = 6 months | 10 | 82 | (80-86) | 10 | 51 | (30-61) |
| | 25° C./60% RH | 15 | 90 | (88-93) | 15 | 62 | (53-70) |
| | | 30 | 91 | (88-94) | 30 | 68 | (62-74) |
| | | 45 | 90 | (89-92) | 45 | 70 | (66-76) |
| | | 60 | 91 | (88-93) | 60 | 85 | (83-88) |
| | | (ramp) | | | (ramp) | | |
| | T = 6 months | 10 | 24 | (22-27) | 10 | 81 | (80-84) |
| | 40° C./75% RH | 15 | 61 | (56-67) | 15 | 86 | (85-88) |
| | | 30 | 69 | (65-75) | 30 | 87 | (85-89) |
| | | 45 | 79 | (68-87) | 45 | 87 | (86-89) |
| | | 60 | 90 | (88-93) | 60 | 90 | (86-95) |
| | | (ramp) | | | (ramp) | | |

Formulation 5A

Another stability study was initiated for Formulation 5A. The capsules were packaged as per Formulation 1A and Formulation 2A and store under the same conditions.

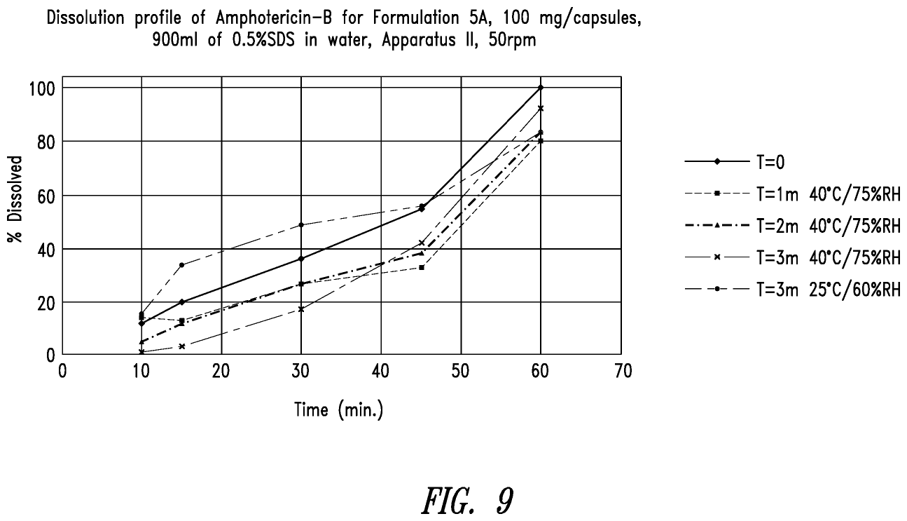

An Amphotericin B/TPGS/Peceol/Gelucire 44/14 semi-solid lipid based formulation in hard shell capsule was prepared (Formulation 5A) and stored under ICH controlled stability conditions. After 3 months of storage, the formulation remained stable with no loss of potency (Table 21) and no increases in related substances (Table 22). The dissolution profile however decreased (Table 23 and FIG. 9). As indicated before, this behavior could be a result a recrystallization or aggregation of the AmpB.

TABLE 21
Stability Results for Formulation 5A.

| Sample | | Formulation 5A |
|---|---|---|
| Formulation | | 11.1% drug load |
| Dose strength | | 100 mg AmpB/caps |
| | T = 0 | Yellow paste in white capsule |
| | T = 1 month | Yellow paste in white capsule (a clear liquid is |
| | 40° C./75% RH | separated in the capsule) |
| Appearance | T = 2months | Yellow paste in white capsule (a clear liquid is |
| | 40° C./75% RH | separated in the capsule and a liquid is observed |
| | | exuding the capsule) |
| | T = 3 months | Yellow paste in white capsule |
| | 25° C./60% RH | |
| | T = 3 months | Yellow paste in white capsule (a liquid is observed |
| | 40° C./75% RH | exuding the capsule) |
| Water content | T = 0 | 1.9% |
| | T = 1 month | 5.1% |
| | 40° C./75% RH | |
| | T = 2months | 2.3% |
| | 40° C./75% RH | |
| | T = 3 months | 2.0% |
| | 25° C./60% RH | |
| | T = 3 months | 2.2% |
| | 40° C./75% RH | |
| Assay | T = 0 | 95.1% |
| % Label claimed | | (n = 6: 94.9, 95.8, 93.7, 96.2, 95.4, 94.6) |
| Corealis -26801-AD-01 | T = 1 month | 97.0% |
| Rev R&D 06 | 40° C./75%RH | (n = 6: 97.4, 97.6, 98.1, 96.6, 95.0, 97.4) |
| | T = 2months | 95.2% |
| | 40° C./75%RH | (n = 6: 96.7, 97.1, 93.9, 96.3, 94.2, 92.7) |
| | T = 3 months | 99.1%^{A} |
| | 25° C./60%RH | (n = 5: 96.5, 97.5, 101.3, 100.5, 99.8) |
| | T = 3 months | 95.3%^{A} |
| | 40° C./75%RH | (n = 5: 98.6, 95.6, 93.6. 97.2. 91.5) |

^{A}Not enough sample units available for n = 6

TABLE 23
Dissolution Profile Stability Results for Formula 5A

| Sample | | Formula 5A | | |
|---|---|---|---|---|
| Dose strength | | 100 mg/capsule | | |
| | | Time (min.) | % dissolved | Range |
| Dissolution 900 ml 0.5% SDS in | T = 0 | 10 | 12 | 2, 22 |
| Water paddles at 50 rpm % dissolved | | 15 | 20 | 12,28 |
| Corealis -26801-B-01 Rev R&D 02 | | 30 | 36 | 28,44 |
| (T = 0, n = 2 | | 45 | 55 | 52,57 |
| T = 1 m, 2 m, n = 3) | | 60 | 101 | 98, 103 |
| (T = 3 m, n = 3) | | (ramp) | | |
| | | Time (min.) | % dissolved | Range^{C} |
| | T = 1 month | 10 | 14 | 2, 24, 15 |
| | 40° C./75% RH | 15 | 13 | 5, 16, 17 |
| | | 30 | 27 | 20, 28, 32 |
| | | 45 | 33 | 30, 35, 35 |
| | | 60 | 81 | 81, 81, 80 |
| | | (ramp) | | |
| | T = 2 months | 10 | 5 | 1, 4, 11 |
| | 40° C./75% RH | 15 | 12 | 10, 10, 17 |
| | | 30 | 27 | 18, 21, 41 |
| | | 45 | 38 | 24, 34, 57 |
| | | 60 | 84 | 90. 83, 78 |
| | | (ramp) | | |
| | T = 3 months | 10 | 15 | 27, 7, 10 |
| | 25° C./60% RH | 15 | 34 | 52, 26, 24 |
| | | 30 | 49 | 63, 49, 35 |
| | | 45 | 56 | 70, 53, 44 |
| | | 60 | 84 | 92, 87, 74 |
| | | (ramp) | | |
| | | Time (min.) | % dissolved | Range^{C, D} |
| | T = 3 months | 10 | 1 | 1, 1, 2 |
| | 40° C./75% RH | 15 | 3 | 1, 3, 6 |
| | | 30 | 17 | 22, 16, 13 |
| | | 45 | 42 | 41, 49, 37 |
| | | 60 | 93 | 90, 94, 95 |
| | | (ramp) | | |

^{C}After 45 minutes at 50 rpm, pieces of the capsule and it contents remain in the sinker.
^{D} The capsules disintegrated slowly compared to the samples at 40° C./75% RH (3 months).

### Example 8. Pharmacokinetics of Formulation 1A and Formulation 5A

The pharmacokinetics of Formulation 1A and Formulation 5 were evaluated in Beagle dogs following a single oral dose and compared to a liquid formulation (i.e., the iCo/Wasan liquid formulation described above). The tissue distribution of these amphotericin B capsule formulations at 24 hours following three days of once a day repeated oral dosing in Beagle dogs was also evaluated.

Amphotericin B in Formulation 1A, Formulation 5A, and in the liquid formulation (i.e., the iCo/Wasan formulation) was administered to male dogs as outlined in the Table 24 below:

TABLE 24
Beagle Dog Study Design

| | | | Dose of | | | |
|---|---|---|---|---|---|---|
| Dose | Dose | Administration | Amphotericin | Dosing | Number | |
| Group | Formulation | Route | B | Days | of Dogs | Comments |
| 1 | Formulation | Oral, single | One capsule of | Day 1 and | 6 | Total of 4 doses |
| | 1A | dose per day | 100 mg per dog | Days 4-6 | | administered (PK |
| | Capsule) | | | | | on plasma samples |
| 2 | Formulation | Oral, single | One capsule of | Day 1 and | 6 | after dosing on |
| | 5A | dose per day | 100 mg per dog | Days 4-6 | | Day 1 and 24 hrs |
| | Capsule) | | | | | after the 4th dose); |
| | | | | | | tissue distribution |
| | | | | | | 24 hrs after the 4th |
| | | | | | | dose. |
| 3 | iCo/Wasan, | Oral, single | 20 ml (100 mg) | Day 1 | 3 | PK on plasma |
| | Liquid | dose | per dog | | | samples after |
| | Formulation | | | | | dosing on Day 1. |
| | (5 mg/mL | | | | | No tissue |
| | of Amp. B) | | | | | distribution. |

Blood was collected for TK evaluation on Day 1, Day 2 and Day 3 (up to 72 hours post-dosing). Dogs receiving the capsule formulations subsequently received a single oral dose for three more days (Days 4-6) and 24 hrs following the last dose the dogs were euthanized and the following tissue samples (approximately 1 g, with exception of mesenteric lymph node) were collected: brain (cerebrum, cerebellum, medulla), heart, kidney (cortex and medulla), liver, lung, spleen, testes, mesenteric lymph node and gastrointestinal tract tissues (duodenum, jejunum, ileum and colon). A sample of intestinal contents was also collected. Plasma samples and tissues were analyzed for amphotericin B content using a qualified LC/MS/MS analytical method.

Oral administration of amphotericin B at a dose of 100 mg in all formulations was well tolerated in dogs and there were no relevant adverse clinical signs observed.

Following oral dosing with amphotericin B in three different formulations, mean plasma levels of amphotericin B initially rose rapidly and in a similar manner (up to 2 hrs post-dosing) and then at a slower rate to attain a plateau (6-24-hrs post-dosing) and declined slowly thereafter. The pharmacokinetic parameters (±SE) determined in all dogs for the Wasan formulation, Formulation 1A, and Formulation 5A following a single dose of amphotericin B are summarized in Table 25 below. The mean Cₘₐₓ, Tₘₐₓ, AUC_{0-Tlast} and MRT_{Last} values were not significantly different from each other for the three formulations.

TABLE 25
Pharmacokinetics

| | | Cₘₐₓ | Tₘₐₓ | AUC_{0-Tlast} | MRT_{Last} |
|---|---|---|---|---|---|
| Formulation | | (ng/mL) | (hr) | (ng*hr/mL) | (hr) |
| Wasan | Mean | 57.4 | 8.0 | 2879 | 31.7 |
| | SE | 2.6 | 2.3 | 128 | 0.4 |
| 1A | Mean | 46.4 | 14.0 | 1700 | 26.7 |
| | SE | 7.1 | 4.5 | 291 | 0.6 |
| 5A | Mean | 52.5 | 8.3 | 2146 | 27.3 |
| | SE | 7.2 | 3.3 | 369 | 1.8 |

Blood plasma concentrations of amphotericin B were measured following administration of Formulation 1A, Formulation 5A, and an oral, liquid dosage form (i.e., the iCo/Wasan formulation described above). These results are reported in Table 26.

TABLE 26
Blood plasma concentrations of amphotericin B in beagle dogs

| | | | | Time Points | | | | | | | | | |
|---|---|---|---|---|---|---|---|---|---|---|---|---|---|
| | | | | pre-dose | 0.5 hr | 1 hr | 2 hrs | 4 hrs | 8 hrs | 12 hrs | 24 hrs | 48 hrs | 72 hrs |
| Day# | Group | Dog ID# | | Final Conc. (ng/ml)* | | | | | | | | | |
| Day 1 | 1 | 001 | DFZ | 0.00 | 8.01 | 0.00 | 16.54 | 43.49 | 34.26 | 23.99 | 51.32 | 19.98 | 9.82 |
| Sep. 21, | Prototype | 002 | CCZ | 0.00 | 7.60 | 11.56 | 22.12 | 24.87 | 21.89 | 19.67 | 22.53 | 9.42 | 8.37 |
| 2016 | 019 | 003 | EGZ | 0.00 | 0.00 | 12.63 | 30.57 | 35.03 | 30.11 | 20.71 | 16.57 | 9.07 | 7.55 |
| | Dose: | 004 | YPZ | 0.00 | 8.14 | 10.75 | 30.82 | 35.20 | 32.56 | 24.16 | 15.36 | 8.64 | 7.37 |
| | Capsule | 005 | ZVZ | 0.00 | 8.60 | 16.51 | 32.47 | 29.68 | 29.52 | 59.47 | 66.43 | 18.94 | 9.84 |
| | | 006 | CJZ | 0.00 | 0.00 | 7.93 | 14.97 | 41.49 | 44.59 | 52.92 | 65.79 | 17.14 | 9.32 |
| | | Mean | | 0.00 | 5.39 | 9.89 | 24.58 | 34.96 | 32.16 | 33.49 | 39.66 | 13.86 | 8.71 |
| | | SE | | 0.00 | 1.71 | 2.28 | 3.16 | 2.86 | 3.03 | 7.27 | 9.92 | 2.19 | 0.45 |
| | 2 | 007 | COZ | 0.00 | 0.00 | 0.00 | 11.75 | 36.53 | 38.23 | 30.24 | 29.48 | 12.27 | 7.96 |
| | Prototype | 008 | DJZ | 0.00 | 8.12 | 11.03 | 24.24 | 35.03 | 36.73 | 30.48 | 27.14 | 12.52 | 9.81 |
| | 022 | 009 | AIZ | 0.00 | 0.00 | 31.44 | 42.60 | 51.37 | 39.87 | 37.42 | 40.71 | 30.87 | 0.00 |
| | Dose: | 010 | BHZ | 0.00 | 0.00 | 29.70 | 33.65 | 42.95 | 39.35 | 53.82 | 85.18 | 42.21 | 31.47 |
| | Capsule | 011 | BIZ | 0.00 | 0.00 | 31.71 | 45.33 | 54.75 | 48.79 | 41.73 | 43.75 | 34.00 | 29.46 |
| | | 012 | EHZ | 0.00 | 29.78 | 32.91 | 48.60 | 44.94 | 44.77 | 38.88 | 34.61 | 30.08 | NPD |
| | | Mean | | 0.00 | 6.32 | 22.80 | 34.36 | 44.26 | 41.29 | 38.76 | 43.48 | 26.99 | 15.74 |
| | | SE | | 0.00 | 4.88 | 5.66 | 5.79 | 3.20 | 1.86 | 3.55 | 8.73 | 4.94 | 5.70 |
| | 3 | 013 | CEZ | 29.79 | 29.41 | 30.10 | 37.82 | 46.77 | 62.28 | 60.90 | 51.29 | 33.66 | 30.12 |
| | Original, | 014 | CPZ | 0.00 | 0.00 | 30.04 | 40.04 | 53.22 | 51.96 | 42.58 | 42.41 | 30.30 | 29.48 |
| | Liquid | 015 | DDZ | 0.00 | 29.22 | 29.53 | 39.95 | 45.99 | 52.81 | 56.77 | 49.37 | 32.87 | 29.47 |
| | Dose: 75 | Mean | | 4.96 | 16.60 | 25.17 | 34.43 | 39.73 | 42.50 | 40.24 | 38.32 | 26.47 | 22.10 |
| | mg/kg | SE | | 7.02 | 8.23 | 5.85 | 8.54 | 10.50 | 12.23 | 11.71 | 9.03 | 6.24 | 6.34 |
| day 7 | 1 | 001 | DFZ | | | | | | | | 40.63 | | |
| Sep. 27, | Prototype | 002 | CCZ | | | | | | | | 71.61 | | |
| 2016 | 019 | 003 | EGZ | | | | | | | | 48.32 | | |
| | Dose: | 004 | YPZ | | | | | | | | 57.45 | | |
| | | 005 | ZVZ | | | | | | | | 38.17 | | |
| | | 006 | CJZ | | | | | | | | 80.08 | | |
| | | Mean | | | | | | | | | 56.04 | | |
| | | SE | | | | | | | | | 6.93 | | |
| | | 001 | COZ | | | | | | | | 54.58 | | |
| | 2 | 002 | DJZ | | | | | | | | 39.12 | | |
| | Prototype | 003 | AIZ | | | | | | | | 49.43 | | |
| | 022 | 004 | BHZ | | | | | | | | 56.72 | | |
| | Dose: | 005 | BIZ | | | | | | | | 70.65 | | |
| | | 006 | EHZ | | | | | | | | 43.29 | | |
| | | Mean | | | | | | | | | 52.30 | | |
| | | SE | | | | | | | | | 4.56 | | |

NPD = No Peak Detected
*All concentrations shown are below the LLOQ (100 ng/mL)

The limit of quantitation was targeted to be 100 ng/mL and a bioanalytical method was established using this limit of quantitation. For all dogs following a single dose, and 24 hrs following repeated dosing of the formulations, no plasma levels above the limit of quantitation, 100 ng/mL were observed.

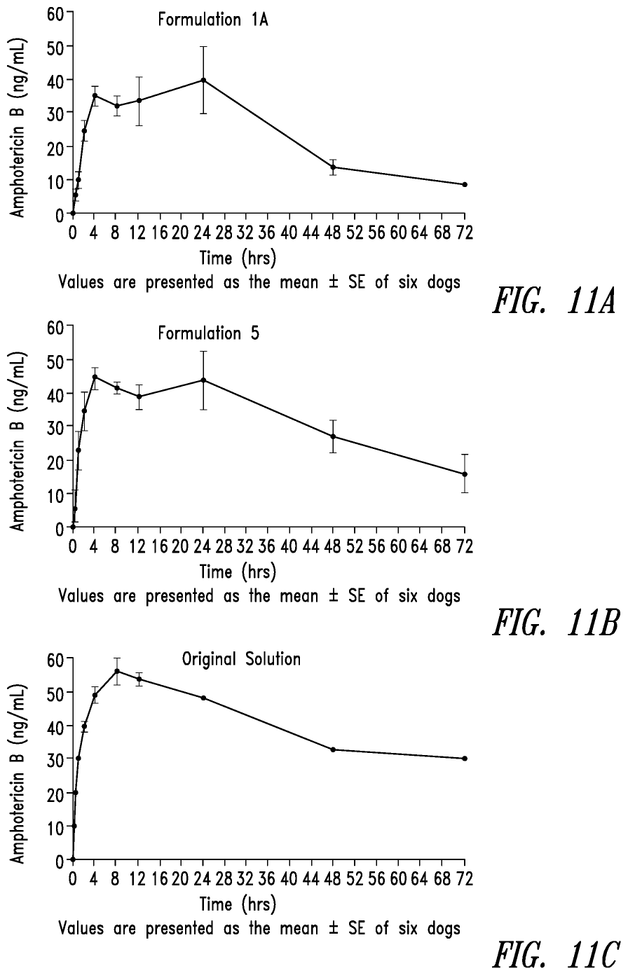

The plasma concentrations of amphotericin B 24 hrs following dosing with the Formulation 1A and Formulation 5A were 56.0±6.9 ng/mL and 52.3±4.6 ng/mL, respectively (See also FIG. 11).

In order to verify that there was no stability issue with plasma samples stored frozen, one dog was dosed with a single capsule (Formulation 1A) and took blood samples at 2 and 4 hrs post-dosing and measured amphotericin B in fresh and frozen samples. The levels of amphotericin B were similar in frozen and fresh samples and also in the same range as the plasma levels presented above.

As an additional part of the investigation, the literature was surveyed for pharmacokinetic studies with amphotericin. Two reports were found. Quantification of amphotericin B was by both HPLC and LC-MS with the lower limit of quantification set at 20 ng/mL. In the first publication (S. Kalbag et al, Cambridge (CAmB)-Focus-Tox-Poster, April 2013), dogs (male and female) were orally dosed with formulated amphotericin B at doses of 15, 30 and 45 mg/kg; at 15 mg/kg, close to the dose in this study (˜10 mg/kg), the plasma Cₘₐₓ ranged from 51.9-67.3 ng/mL (males-females) values close to what was observed in this study and with similar plasma concentration profiles out to 24 hrs. In a study conducted in rats following oral administration of amphotericin B in a novel lipid formulation (E. K. Wasan co-author, J. Antimicrobial Chemotherapy 64:101-108, 2009), the Cmax was 96 ng/mL following a dose of 10 mg/kg, the plasma levels being in the range of what we observed with the original solution. Thus, based on the investigation and the plasma profiles obtained, that plasma concentrations that were measured between the limit of detection and limit of quantification are representative of the actual plasma concentrations obtained.

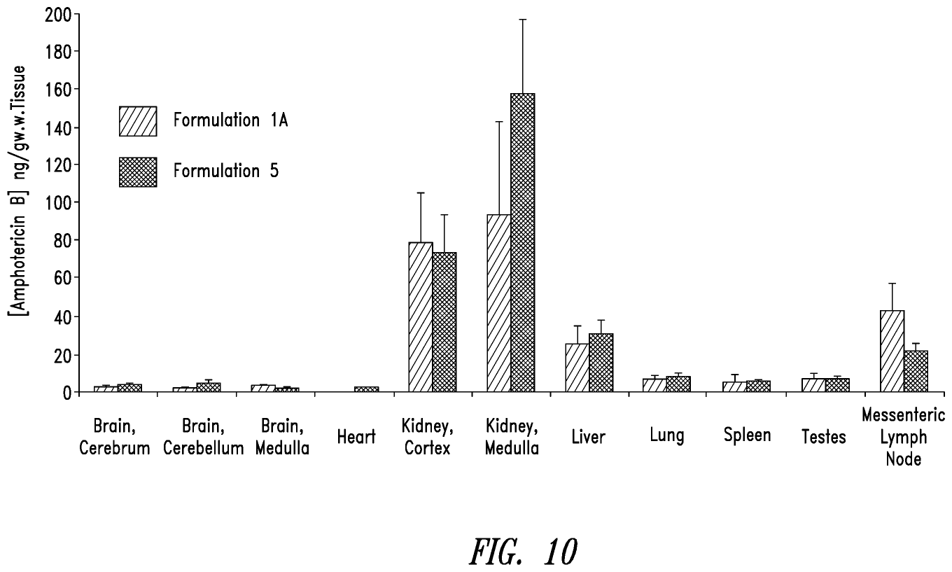

Tissue concentrations of amphoteric B in beagle dogs were measured after administration of dosage forms Formula 1A and Formula 5 in a good laboratory practices (GLP)/dose range finding (DRF) study. Tables 27 shows the tissue levels of amphotericin B (See also FIG. 10)

TABLE 27
Tissue concentration of amphotericin B in a dog model.

| | Mean | SE | | Mean | SE | |
|---|---|---|---|---|---|---|
| | ng/g w.w. tissue | | Kp¹ | ng/g w.w. tissue | | Kp¹ |
| | Prototype | | | Prototype | | |
| Organ | Formulation 019 | | | Formulation 022 | | |
| Brain, Cerebrum | 2.9 | 1.1 | 0.05 | 4.0 | 1.2 | 0.08 |
| Brain, Cerebellum | 2.3 | 0.8 | 0.04 | 5.0 | 1.8 | 0.10 |
| Brain, Medulla | 3.6 | 0.9 | 0.06 | 2.8 | 0.6 | 0.05 |
| Heart | 0.0 | 0.0 | 0.00 | 2.5 | 0.7 | 0.05 |
| Kidney, Cortex | 78.3 | 27.1 | 1.40 | 72.9 | 21.0 | 1.40 |
| Kidney, Medulla | 93.8 | 49.5 | 1.67 | 157.8 | 40.0 | 3.03 |
| Liver | 25.4 | 10.4 | 0.45 | 30.9 | 7.9 | 0.59 |
| Lung | 7.2 | 2.3 | 0.13 | 8.7 | 1.8 | 0.17 |
| Spleen | 5.3 | 4.3 | 0.10 | 6.0 | 1.6 | 0.12 |
| Testes | 7.4 | 2.6 | 0.13 | 7.6 | 1.4 | 0.15 |
| Messenteric Lymph | 42.9 | 14.6 | 0.77 | 21.7 | 4.8 | 0.42 |
| Duodenum | 69.3 | 32.2 | 1.24 | 69.0 | 31.7 | 1.33 |
| Jejunum | 533.7 | 174.9 | 9.52 | 382.1 | 256.9 | 7.34 |
| Ileum | 422.3 | 142.4 | 7.54 | 346.7 | 165.6 | 6.66 |
| Colon | 703.3 | 427.5 | 12.55 | 481.3 | 131.2 | 9.25 |
| Intestinal Contents | 1938.8 | 785.4 | n.r. | 3106.6 | 1235.9 | n.r. |

Concentration value is below the LLOQ (<11.00 ng/mL)
Concentration value is above the ULOQ (>550 ng/mL)
A Dilution factor of 11 was applied to all final reported concentration values
NPD: No peak detected
NRV: No reportable value. Overly high analyte peak response was detected, but no concentration value could be calculated by software.
Values are presented as the mean ± SE of n = 6 and included plasma concentrations above the limit of detection but below the limit of quantification and values above the upper limit of quantification; samples with no peak detected were included in the means as values of zero.
¹Kp, the tissue partition coefficient, was calculated by dividing the mean tissue levels by the mean plasma levels of amphotericin B observed following repeated dosing with prototype Formulation 1A and Formulation 5A and assuming that 1 g of tissue represents 1 mL of tissue volume.

The distribution of amphotericin B amongst tissues and intestinal contents was similar following repeated dosing with amphotericin B in formulations Formulation 1A and Formulation 5. Gastrointestinal tissues and contents contained the highest levels with intestinal content levels ranging from 1938.8-3106.6 ng/g w/w. of sample and tissue levels and tissue/plasma ratios ranging from 69.0-703.3 ng/g w/w. tissue and 1.24-12.55, respectively. Amongst non-gastrointestinal tissues, the kidney cortex and medulla followed by the liver and mesenteric lymph node had the highest levels with tissue levels and tissue/plasma ratios ranging from 21.7-157.8 ng/g w/w. and 0.42-3.03, respectively. The remaining tissues had very low levels of amphotericin B, with tissue levels and tissue/plasma ratios ranging from 0.0-7.6 ng/g w.w. and 0.00-0.17 ng/g w.w., respectively.

In conclusion, oral dosing of 100 mg amphotericin B contained in Formulation 1A, Formulation 5, and the liquid formulation was well tolerated in dogs. The oral bioavailability of amphotericin B from iCo-010 (iCo/Wasan liquid formulation) and Formulation 1A and Formulation 5 (capsule formulations) were similar with no significant differences noted between the formulation groups for Cₘₐₓ, Tₘₐₓ and AUC_{0-Tlast}. The tissue distribution of amphotericin B following dosing with Formulation 1A and Formulation 5 was similar, with the highest levels found in gastrointestinal tissues followed by the kidney, liver and mesenteric lymph node, lower levels observed in the lung, spleen and testis and very low levels observed in regions of the brain and the heart.

### Example 9. Pharmacokinetics of Amphotericin B Following Oral Administration to Fed and Fasted Beagle Dogs

This study involved dosing the test item by the oral route over two different periods and in a cross-over design as outlined in Table 28.

TABLE 28
Study Outline

| | | Dose of | | Wash-Out and | |
|---|---|---|---|---|---|
| | # of | Amphotericin B | | Observation | |
| Sequence | Dogs | (mg) | Period 1 | Period | Period 2 |
| I | 2M | 500 | Fed | 7-Days | Fasted |
| II | 2M | 500 | Fasted | 7-Days | Fed |

M = male

Administration of Test Items

The test item capsules were administered on each dose period as follows.

Fasted Dosing:

Dogs were fasted overnight. Dosing commenced the next morning at approximately 8:30-9:00 a.m. Five (5) capsules, each containing 100 mg of formulated amphotericin B, were dosed orally by placing one at a time on the back of the tongue. Immediately following dosing of the last capsule, ˜20 mL of tap water was administered slowly via syringe in the corner of the mouth to ensure swallowing. The dog's mouth was checked again to ensure that there was no evidence of capsules in the mouth. Food (Lab Diet Certified Canine Diet #5007) was administered following the 2 hrs blood sampling time. Water was provided ad libitum.

Fed Dosing:

Dogs were fasted overnight. Dosing commenced the next morning at approximately 8:30-9:00 a.m. Five (5) capsules, each containing 100 mg of formulated amphotericin B, were dosed orally by placing one at a time on the back of the tongue. Immediately following dosing, ˜20 mL of tap water was administered slowly via syringe in the corner of the mouth to ensure swallowing. The dog's mouth was checked again to ensure that there is no evidence of capsules in the mouth and 300 (±5) grams of moist dog food (Pedigree® Meaty Loaf with Real Chicken) was offered from a food bowl. All dogs consumed the 300±5 grams of canned wet dog food within 4-5 minutes. Water was provided ad libitum.

In-Life Observations

Mortality.

Mortality checks were performed and documented twice daily on dosing days and once daily on non-dosing days during the study period. On the dosing day, animals were monitored closely for the first 60 minutes after dosing.

Body Weights:

Body weights were recorded prior to each dose and at the end of the 7-day observation period.

Blood Sampling for Pharmacokinetics:

Blood samples were collected from all animals following dosing as followings: on dosing days 1 and 8, blood samples were collected 0.5 hr, 1 hr, 2 hrs, 4 hrs, 6 hrs, 12 hrs, and 24 hrs post dosing.

For the purpose of collection of the samples indicated above, each anima was bled from the jugular vein. Each blood sample (approximately 2 mL) was collected into a vacutainer tube containing an anticoagulant (K2EDTA). The time (actual time, in conjunction with the day and time of dosing) was recorded for each sample.

Following the collection, the blood was placed in a refrigerated centrifuge for 20 minutes at 2000 rpm in order to separate the plasma. The recovered plasma was stored in duplicate vials and frozen (at −80±10° C.) pending analysis. For each step in preparation of plasma, the samples were, as much as possible, protected from ambient light.

Following the final blood collection time each animal was returned to the Nucro-Technics' dog colony.

Sample Analysis:

Plasma sample analysis was performed at Nucro-Technics' Bioanalytical Laboratory using a qualified LC-MS/MS method for the determination of amphotericin B. Plasma samples will be retained for three months after the final report has been issued.

Pharmacokinetic Analysis:

Plasma concentration-time data was analyzed by the non-compartmental method to obtain the pharmacokinetic parameters using validated Phoenix® WinNonlin® version 6.3 software (Pharsight Corp).

The main parameters (listed below) were calculated:

AUC_{0-Tlast}: Area under the plasma concentration-time curve from time zero to the time of the last quantifiable concentration at time tlast, calculated using the linear trapezoidal rule.

AUC_{0-∞}: Area under the plasma concentration curve from time zero extrapolated to infinity. AUC_{0-∞} was calculated as AUC_{0-Tlast}+(Cₗₐₛₜ/kₑ)

Cₘₐₓ: Maximum plasma concentration

Tₘₐₓ: Time of maximum concentration determined from the nominal time of blood sampling

kₑ: Elimination rate constant. This was estimated using linear regression on the terminal phase of the semi-logarithmic concentration-time curve. A minimum of three data points will be used for the calculation of kₑ. No weighting was applied to the regression line.

t_{1/2(e)}: Terminal elimination half-life calculated from ln(2)/kₑ

Additional parameters such as mean residence time (MRT); clearance after oral dosing (CL/F), and volume of distribution/after oral dosing (Vz/F) generated by the software may be reported at the discretion of the Study Director.

Statistical Analysis:

AUC_{0-Tlast}, and Cₘₐₓ were used as primary outcome variables to compare bioavailability between the fed and fasted states and Tₘₐₓ was considered for absorption. Significant differences between the two formulation groups with respect to AUC_{0-Tlast}, Cₘₐₓ and Tₘₐₓ were assessed using a Student's t-test and employing the p<0.05 level as an indication of statistically significant differences between the two formulation groups. Significant differences between the variances of grouped data were assessed by using an F-test for two groups and accepting the p<0.05 level as an indication of significant differences between the variances.

Results

Clinical Observations

Oral administration of amphotericin B formulated as Formulation 1A at a dose of 500 mg (contained in five capsules) was well tolerated in dogs and there were no relevant adverse clinical signs observed. Body weight was maintained throughout the treatment period (Table 29).

TABLE 29
Summary of Body Weights

| Body Weight (kg) | |
|---|---|
| Period 1 Study Day 1 | Period 2 Study Day 8 |

| Dog ID | Fed | Fasted | Fed | Fasted |
|---|---|---|---|---|
| 001 XSP | 9.0 | — | — | 9.2 |
| 002 KKR | 9.7 | — | — | 9.5 |
| 003 VSR | — | 8.9 | 9.1 | — |
| 004 WHP | | 91 | 9.2 | |

Pharmacokinetics in Fasted and Fed Dogs

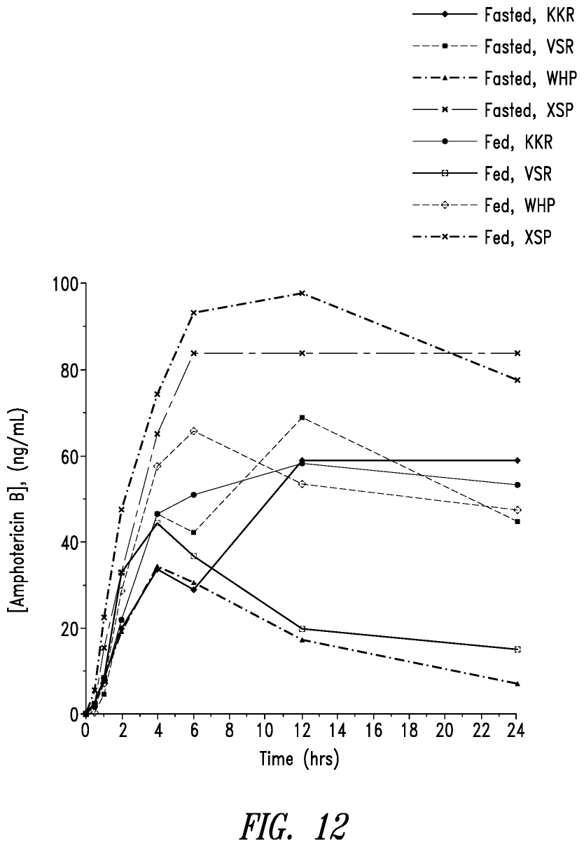

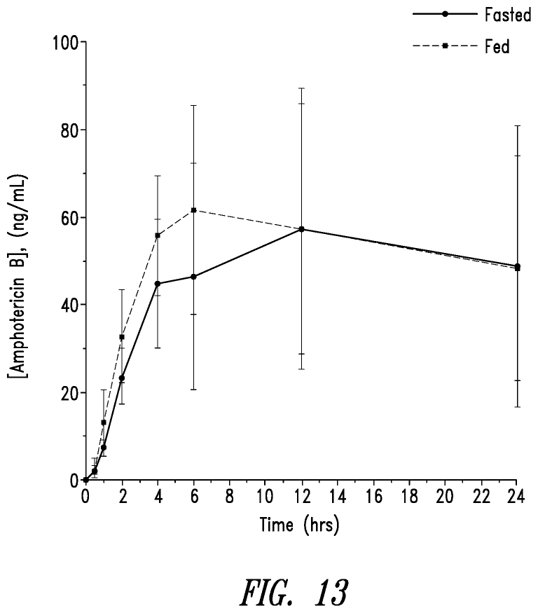

The mean plasma concentrations of amphotericin B are presented in Table 30 and the individual and mean plasma concentrations versus time profiles of amphotericin are presented in FIGS. 12 and 13. The pharmacokinetic parameters derived from the plasma concentration versus time profiles are presented in Table 31.

Following oral dosing with amphotericin B formulated as Formulation 1A, mean plasma levels of amphotericin B initially rose rapidly and in a similar manner (up to 2 hrs post-dosing) and then at a slower rate to attain either a plateau (6-24 hrs post-dosing) or peak at 4 hrs post-dosing and decline thereafter. In most cases, the elimination phase was poorly defined resulting in an in ability to determine the terminal elimination phase pharmacokinetic parameters. A review of the pharmacokinetic parameters presented in Table 31 indicates that the mean Cₘₐₓ, Tₘₐₓ and AUC_{0-Tlast} values were not significantly different from each other for fasted and fed states. One different dog in each of the fasted and fed groups had a lower AUC_{0-Tlast} by virtue of a substantial drop in the plasma concentrations of amphotericin B at 4 hrs post-dosing. The variation in the pharmacokinetic parameters reported was not different between the fasted and fed groups.

The lack of a significant difference between the pharmacokinetic parameters for the fasted and fed states and their variances suggests that the presence of food has little impact on the absorption of amphotericin B from formulation iCo-019.

TABLE 30
Plasma Concentrations of Amphotericin B Following Oral
Dosing with Formulation 1A in Fasted and Fed Dogs.

| Fasted | Fed |
|---|---|
| (=4) | (n = 4) |

| | | | | |
|---|---|---|---|---|
| Time | Mean | SD | Mean | SD |

| | | | | |
|---|---|---|---|---|
| (hr) | (ng/mL) | CV % | (ng/mL) | CV % |

| | | | | | | |
|---|---|---|---|---|---|---|
| 0 | 0.00 | 0.00 | n.a. | 0.00 | 0.00 | n.a. |
| 0.5 | 1.91 | 1.31 | 69 | 2.53 | 2.32 | 92 |
| 1 | 7.21 | 1.90 | 26 | 13.04 | 7.51 | 58 |
| 2 | 23.61 | 6.38 | 27 | 32.75 | 10.66 | 33 |
| 4 | 44.84 | 14.72 | 33 | 55.75 | 13.72 | 25 |
| 6 | 46.37 | 25.87 | 56 | 61.66 | 23.89 | 39 |
| 12 | 57.36 | 28.61 | 50 | 57.30 | 31.85 | 56 |
| 24 | 48.66 | 32.07 | 66 | 48.34 | 25.62 | 53 |

This data is presented as mean ± SE of n = 4; n.a.—not applicable.

TABLE 31
PK Parameters of Amphotericin B Following Oral
Dosing of 500 mg Amphotericin B Formulated
as Formulation 1A in Fasted and Fed Dogs.

| Feeding State | Dog | Cₘₐₓ | Tₘₐₓ | AUC_{0-Tlast} | MRT_{Last} |
|---|---|---|---|---|---|
| Fasted | KKR | 58.95 | 12 | 1104 | 14.37 |
| | VSR | 69.00 | 12 | 1188 | 12.95 |
| | WHP | 34.26 | 4 | 424 | 9.32 |
| | XSP | 84.19 | 6 | 1785 | 13.34 |
| | Mean | 61.60 | 9 | 1125 | 12.49 |
| | SD | 20.97 | 4 | 557 | 2.20 |
| | % CV | 34 | 49 | 50 | 18 |
| Fed | KKR | 58.07 | 12 | 1177 | 13.25 |
| | VSR | 44.46 | 4 | 572 | 10.00 |
| | WHP | 65.74 | 6 | 1191 | 12.34 |
| | XSP | 97.71 | 12 | 1956 | 12.63 |
| | Mean | 66.50 | 9 | 1224 | 12.06 |
| | SD | 22.59 | 4 | 567 | 1.42 |
| | % CV | 34 | 49 | 46 | 12 |

None of the statistical parameters and their variances were significantly different between the fasted and fed states.

### CONCLUSIONS

In conclusion, the oral capsule dosing of 500 mg amphotericin B as Formulation 1A was well tolerated in dogs. The Cₘₐₓ, Tₘₐₓ, AUC_{0-Tlast} and MRTₗₐₛₜ in fasted and fed dogs were not considered significantly different. Therefore, this study demonstrated that the presence of food does not affect the oral absorption of amphotericin B from Formulation 1A in Beagle dogs.

1. A solid dosage form comprising:
amphotericin B, and
at least one lipophilic component;
coated on a solid carrier.

2. The solid dosage form of claim 1, wherein a % w/w of amphotericin B in the solid dosage form is greater than a % w/w of the at least one lipophilic component.

3. The solid dosage form of claim 1, wherein the % w/w of amphotericin B is in the range of about 20% to about 30% of the total weight of the solid dosage form.

4. The solid dosage form of claim 1, wherein amphotericin B is present in an amount in the range of from about 50 mg to about 200 mg.

5. The solid dosage form of any of claim 1, wherein amphotericin B is present in amount of about 100 mg.

6. The solid dosage form of claim 1, wherein the amphotericin B is present in amount of about 150 mg.

7. The solid dosage form of claim 1, wherein the at least one lipophilic component is selected from the group consisting of a polyethylene oxide-containing fatty acid ester, fatty acid glycerol ester, and combinations thereof.

8. The solid dosage form of claim 1, wherein the solid carrier is a bead or a saccharide.

9. The solid dosage form of claim 1, wherein the Cₘₐₓ of amphotericin B is within the range of from about 80% to about 125% of the Cₘₐₓ of amphotericin B measured after oral administration of a liquid formulation having an equivalent dose of amphotericin B.

10. The solid dosage form of claim 1, wherein the AUC₀₋₂₄ of amphotericin B is within the range of from about 80% to about 125% of the AUC₀₋₂₄ of amphotericin B measured after oral administration of a liquid formulation having an equivalent dose of amphotericin B.

11. The solid dosage form of claim 1, wherein the AUC₀₋₄₈ of amphotericin B is within the range of from about 80% to about 125% of the AUC₀₋₄₈ of amphotericin B measured after oral administration of a liquid formulation having an equivalent dose of amphotericin B.

12. The solid dosage form of claim 1, wherein the Tₘₐₓ of amphotericin B is within the range of from about 80% to about 125% of the Tₘₐₓ of amphotericin B measured after oral administration of a liquid formulation having an equivalent dose of amphotericin B.

13. A capsule comprising the solid dosage form of claim 1.

14. A method of treating leishmaniasis in a subject in need thereof comprising administering to the subject an effective amount of the solid dosage form of claim 1.
